(19) United States
(12) Patent Application Publication
(10) Pub. No.: US 2023/0301807 A1
(43) Pub. Date: Sep. 28, 2023

(54) A CLOT MOBILIZER DEVICE FOR EXTRACTION OF AN OCCLUSION FROM A BLOOD VESSEL

(71) Applicant: ANACONDA BIOMED, SL, Sant Quirze del Vallès (ES)

(72) Inventors: Daniel GARCIA SABIDO, Vilafranca del Penedès (ES); David FERNÁNDEZ SÁNCHEZ, Sabadell (ES); Ane LIZARAZU GONZALEZ, Barcelona (ES); Iñaki GALVE MURILLO, Barcelona (ES); Ofir ARAD HADAR, Sant Cugat del Vallès (ES); Francesc JARA MUNS, Barcelona (ES); Alizée PACE, Mies Vaud (CH)

(21) Appl. No.: 18/040,492

(22) PCT Filed: Jul. 14, 2021
(86) PCT No.: PCT/EP2021/069612

§ 371 Date: Feb. 3, 2023

(30) Foreign Application Priority Data
Aug. 3, 2020 (EP) 20382718.3
Aug. 3, 2020 (EP) 20382719.1

(51) Int. Cl. A61F 2/86

(52) U.S. Cl. CPC A61F 2/86; A61F 2002/825

(57) ABSTRACT

A clot mobilizer device for extraction of an occlusion from a blood vessel is provided, which comprises a working portion including a plurality of crowns of cells. Each cell comprises an open area bordered by struts. A distal end of each cell in a first crown is contiguous with a proximal end of a corresponding cell in a third crown, and a distal end of each cell in a second crown is contiguous with a proximal end of a corresponding cell in a fourth crown. The second crown is disposed distal to the first crown and proximal to the third crown, and the fourth crown is disposed distal to the third crown. The plurality of crowns of cells define a tubular-shaped section forming a cylindrically closed structure. The clot mobilizer also comprises a tapered portion extending proximally from the proximal end of the working portion and comprising a plurality of struts.

### TECHNICAL FIELD

The present invention is directed, in general, to the field of intravascular medical devices and in particular, to a clot mobilizer device for extraction of an occlusion from a blood vessel.

### BACKGROUND OF THE INVENTION

Clot mobilizers are self-expanding devices utilized to capture and remove a blood clot or thrombus from a blood vessel. The clot mobilizer is firstly introduced into the blood vessel with a catheter, and then is inserted into the thrombus and deployed. Upon deployment, the clot mobilizer expands to capture the thrombus. The clot mobilizer is removed from the blood vessel allowing blood flow restoration.

A known clot mobilizer is the Solitaire™ X revascularization device manufactured by Medtronic. The Solitaire™ X is designed for use in the flow restoration of patients with ischemic stroke due to large intracranial vessel occlusion. The stent-like design of the Solitaire™ X allows immediate clot retrieval and blood flow restoration. Similar to the clot mobilizer of present invention, the Solitaire™ X comprises an elongated body with a plurality of interconnected struts that in an expanded configuration define an open tubular-shaped section with a pattern defined by almond-shaped cells.

Besides, patent U.S. Ser. No. 10/426,644 provides a medical device for blood flow restoration and/or for use as an implantable member in a human vessel and includes a self-expanding member, a guidewire, and a connection mechanism. The self-expanding member includes a plurality of cells and filaments having specific ranges of thicknesses, widths, and heights. The self-expanding member can take on a volume-reduced coiled form with overlapped edges, and can generate optimal radial forces against a vessel wall and/or thrombus when deployed and expanded. While in a volume-reduced form, the self-expanding member, similar to a wire mesh roll, or piece of paper, is curled up such that its edges overlap to be introduced into and moved within a microcatheter.

Likewise, patent U.S. Pat. No. 8,940,003 discloses methods for restoring blood flow in occluded blood vessels using an apparatus having a self-expandable distal segment that is pre-formed to assume a superimposed structure in an unconstrained condition but can be made to take on a volume-reduced form making it possible to introduce it with a microcatheter and a push wire arranged at the proximal end, with the distal segment in its superimposed structure assuming the form of a longitudinally open tube and having a mesh structure of interconnected strings or filaments or struts. The distal segment is a flat or two-dimensional structure that is rolled up or curled up in such a way that its edges are at least closely positioned to each other and may overlap in the area of the edges. The distal segment can have a tapering structure at its proximal end where the strings or filaments or struts converge at a connection point. The distal segment consists of a mesh or honeycomb structure that comprises a multitude of filaments interconnected by a laser welding technique.

The known devices/apparatus do not comprise a tapered portion having several struts with a width greater than a width of the struts of a working portion. Furthermore, the known devices do not disclose a closed tube or closed structure that is expanded by expanding the individual cells. In particular, regarding the Solitaire™ X this device is formed by a layer comprising two overlapping edges. Likewise, although the Solitaire™ X performs well even through complicated anatomy, for large blood vessels its diameter volume has to be increased. That is, the same device cannot be used in blood vessels of different diameters.

New clot mobilizer devices usable in blood vessels of different diameters are therefore needed.

### DESCRIPTION OF THE INVENTION

To that end, embodiments of the present invention provide, according to one aspect, a clot mobilizer device for extraction of an occlusion from a blood vessel. The clot mobilizer device comprises a working portion having a plurality of crowns (or rows) of cells. Each cell of the working portion includes an open area bordered by struts, where: a distal end of each cell in a first crown of the plurality of crowns is contiguous with a proximal end of a corresponding cell in a third crown of the plurality of crowns, a distal end of each cell in a second crown of the plurality of crowns is contiguous with a proximal end of a corresponding cell in a fourth crown of the plurality of crowns, the second crown is disposed distal to the first crown and proximal to the third crown, the fourth crown is disposed distal to the third crown, first and second opposite midportions of each cell in each of the first, second, third and fourth crowns each are contiguous with a midportion of an adjacent cell in such crown.

According to the embodiments of the invention, the clot mobilizer device also comprises a tapered portion extending proximally from a proximal end of the working portion, the tapered portion also having a plurality of struts. The tapered portion has a smaller diameter at a proximal end than an expanded diameter of the working portion. Moreover, at least some of the tapered portion struts have a width greater than a width of the working portion struts.

Advantageously, in the proposed clot mobilizer the outward radial forces are maintained for a broad range of vessels (as is seen in Example 3), and better pushability of the clot mobilizer device is achieved. This behavior allows lower radial forces in small diameters and larger radial forces in large diameters without, leaving a safety window. Remarkably, due to the fact of having less radial force in small diameters, the risk of damaging the vessel is reduced, in comparison with Solitaire (or other known devices for removing clots). Moreover, the proposed clot mobilizer device can be used in vessels of different diameters (between 1.5-4 mm).

In an embodiment, the clot mobilizer device is self-expandable.

Alternatively, in another aspect, the invention provides a clot mobilizer device for extraction of an occlusion from a blood vessel comprising a plurality of crowns of cells in a working portion, each cell comprising an open area bordered by struts, a distal end of each cell in a first crown of the plurality of crowns being contiguous with a proximal end of a corresponding cell in a third crown of the plurality of crowns, a distal end of each cell in a second crown of the plurality of crowns, being contiguous with a proximal end of a corresponding cell in a fourth crown of the plurality of crowns, the second crown being disposed distal to the first crown and proximal to the third crown, the fourth crown being disposed distal to the third crown, first and second opposite midportions of each cell in each of the first, second, third and fourth crowns each being contiguous with a midportion of an adjacent cell in such crown. The working portion is configured to expand from a compressed diameter of less than 1.5 mm to an expanded diameter of at least 3.5 mm and to exert an outward radial force between 0.75 N and 3 N at every diameter between and including the compressed diameter and the expanded diameter.

According to this aspect, the clot mobilizer device can also comprise a tapered portion extending proximally from a proximal end of the working portion, and comprising a plurality of struts; the tapered portion having a smaller diameter at a proximal end than an expanded diameter of the working portion. Likewise, in an embodiment, the tapered portion struts also have a width greater than a width of the working portion struts.

Moreover, the crowns of cells of the clot mobilizer define a tubular-shaped section forming a cylindrically closed structure. To that effect, the proposed clot mobilizer device can be manufactured by producing specified cuts either on a tube or on a wire.

In an embodiment, the clot mobilizer device comprises a pusher (or pusher wire) manufactured by producing specified cuts on said wire.

In another aspect, the invention provides a thrombectomy system which comprises the clot mobilizer device of the present invention and a microcatheter configured to be advanced through vasculature of a patient to a thrombus site within a blood vessel and adapted to carry the clot mobilizer to the thrombus site; wherein the clot mobilizer is configured to be movably disposed within the microcatheter in a retracted position.

The aspect previously provided can be used for extracting a thrombus from a thrombus site in a blood vessel by following a method comprising the steps of advancing the microcatheter through vasculature toward the thrombus site, wherein advancing the microcatheter comprises advancing a distal end of the microcatheter through the thrombus, the clot mobilizer device being disposed within the microcatheter; advancing the clot mobilizer until reaching the distal end of the microcatheter, moving the microcatheter and the clot mobilizer device with respect to each other to place the clot mobilizer device outside of the microcatheter allowing the clot mobilizer device to expand from the compressed configuration to the expanded configuration. More particularly, the method further comprises the step of self-expanding the clot mobilizer from the compressed configuration to the expanded configuration.

In another aspect, the invention provides a thrombectomy system which comprises the clot mobilizer device of the present invention; a delivery catheter configured to be advanced through vasculature of a patient to a thrombus site within a blood vessel; an aspiration catheter adapted to apply suction to an expandable aspiration funnel extending from a distal end of the aspiration catheter, the aspiration funnel being configured to be movably disposed within the delivery catheter in a retracted position in a compressed state and at least partially outside the delivery catheter in an extended and expanded position, the aspiration funnel comprising a non-permeable covering, a diameter of a distal end of the aspiration funnel being greater in the extended and expanded position than in the retracted position, the aspiration funnel being configured to adapt its shape and length to an inner wall of the blood vessel such that the aspiration funnel reduces (or stops) blood flow through the blood vessel and lengthens as it narrows to retain a thrombus within the aspiration funnel; the clot mobilizer device configured to capture the thrombus and to be at least partially withdrawn with the captured thrombus into the aspiration funnel; and the thrombectomy system further comprises a microcatheter adapted to carry the clot mobilizer device to the thrombus site; wherein the clot mobilizer device is configured to be movably disposed within the microcatheter in a retracted position, and wherein the microcatheter is configured to be movably disposed within the aspiration catheter.

In an embodiment, the delivery catheter, the aspiration funnel, the microcatheter and the clot mobilizer device are oriented on a same axis, and can be coaxially configured and movable to each other.

The aspect previously provided can be used for extracting a thrombus from a thrombus site in a blood vessel by following a method comprising the steps of advancing a delivery catheter through vasculature toward the thrombus site; placing a distal end of the delivery catheter proximal to the thrombus in the blood vessel; advancing the aspiration catheter within the delivery catheter, the aspiration funnel extending distally from the aspiration catheter; expanding the aspiration funnel into contact with inner walls of the blood vessel, thereby reducing (or stopping) blood flow past the aspiration funnel; advancing the clot mobilizer device distally through the aspiration funnel toward the thrombus; deploying the clot mobilizer device to capture the clot; moving the clot mobilizer device and thrombus proximally toward the aspiration funnel; applying suction through the aspiration catheter to the aspiration funnel to aspirate the thrombus at least partially into the aspiration funnel; and moving the aspiration funnel and the thrombus proximally within the vasculature, the aspiration funnel adapting its shape and length to a surrounding blood vessel by lengthening as it narrows to retain the thrombus within the aspiration funnel.

In a particular embodiment, the method further comprises the steps of advancing a microcatheter within the aspiration catheter, wherein advancing the microcatheter comprises advancing a distal end of the microcatheter through the thrombus, the clot mobilizer device being disposed within the microcatheter; moving the microcatheter and the clot mobilizer device with respect to each other to place the clot mobilizer device outside of the microcatheter; and expanding the clot-capture device allowing the clot mobilizer device to self-expand. More particularly, the method further comprises the step of moving the clot mobilizer device proximally at least partially into the aspiration funnel.

In an embodiment, the clot mobilizer is manufactured by providing a tube; having a longitudinal axis therethrough, providing a stationary source of laser radiation, generating a beam of laser radiation using the source of laser radiation, and cutting a desired pattern into the tube by scanning the beam over a desired region of the tube. In another embodiment, the clot mobilizer is manufactured by providing a wire; having a longitudinal axis therethrough, producing predetermined cuts of the cross section of the wire by means of an ultrashort pulse laser in order to produce a predetermined shape of the stent. A suitable manufacturing method is described e.g. in U.S. Ser. No. 10/434,605B2.

In an embodiment, the working portion is configured to have a compressed diameter of less than 1.5 mm and to exert an outward radial force between 1.75 N and 3 N, particularly 2.1 N, when the compressed diameter is around 1.5 mm. In an embodiment, the working portion in a compressed diameter of 1.5 mm is configured to exert an outward radial force between 1.75 N and 3 N, and particularly 2.1 N.

In an embodiment, the working portion is configured to have an expanded diameter of at least 3.5 mm and to exert an outward radial force between 0.75 N and 1.5 N, particularly 1 N, when the expanded diameter is around 3.5 mm. In an embodiment, the working portion in an expanded diameter of 3.5 mm is configured to exert an outward radial force between 0.75 N and 1.5 N, and particularly 1 N.

In an embodiment, the working portion is configured to exert an outward radial force in the range between 0.75 N and 3 N at every diameter between and including a compressed diameter of 1.5 mm and an expanded diameter of 3.5 mm, resulting in a flat curve behavior (as it can be seen in FIG. 8), allowing lower radial forces in small diameters and larger radial forces in large diameters, without, leaving a safety window. Consequently, the outward radial forces are maintained for a broad range of vessels with different diameters and the risk of damaging the vessel is reduced. In a particular embodiment, the working portion is configured to exert an outward radial force in the range, inside a safety window, between 1 N and 2.1 N at every diameter between and including a compressed diameter of 1.5 mm and an expanded diameter of 3.5 mm.

In an embodiment, the cells in the working portion have an almond shape.

In an embodiment, first and second tapered portion struts of the plurality of tapered portion struts converge from a proximal end of the working portion to a distal end of a proximal connection portion to partially define a proximal cell.

In an embodiment, a third tapered portion strut of the plurality of tapered portion struts extends distally from the first tapered portion strut from a point distal to the proximal connection portion and a fourth tapered portion strut of the plurality of tapered portion struts extends distally from the second tapered portion strut from a point distal to the proximal connection portion, the third and fourth tapered struts partially defining the proximal cell.

In an embodiment, the third and fourth tapered portion struts converge at the proximal end of the working portion.

In some embodiments, at least some of the working portion struts can have a width between 30 μm and 60 μm, particularly between 40 μm and 50 μm and more particularly 43 μm or 48 μm.

In some embodiments, at least some of the tapered portion struts can have a width between 60 μm and 155 μm, particularly between 70 μm and 145 μm and more particularly 71 μm or 142 μm.

In some embodiments, the working portion in the expanded diameter can have a length between 20 mm and 50 mm, particularly between 38 mm and 42 mm and more particularly 40 mm.

In some embodiments, the working portion in the compressed diameter can have a length between 10 mm and 60 mm, particularly between 47 mm and 51 mm and more particularly 49 mm.

In some embodiments, the tapered portion can have a length between 10 mm and 20 mm.

In some embodiments, the tapered portion in the expanded diameter can have a length between 10 mm and 20 mm, particularly between 13 mm and 17 mm and more particularly 15 mm.

In some embodiments, the tapered portion in the compressed diameter can have a length between 10 mm and 20 mm, particularly between 10 and 14 mm and more particularly 12 mm.

In an embodiment, the tapered portion struts have a thickness equal to a thickness of the working portion struts.

In an embodiment, the clot mobilizer device further comprises a pusher extending proximally from the proximal connection portion (or first connection portion).

Embodiments of the present invention also provide, according to another aspect, a clot mobilizer device with an improved attachment of their elements. The clot mobilizer device can be advanced distally and withdrawn proximally from a proximal end and rotated about a longitudinal axis from the proximal end. The clot mobilizer device comprises an elongated device (e.g., a stent retriever or other medical device) comprising a first connection portion (or proximal connection portion) extending proximally, the first connection portion having a first attachment surface; a pusher comprising a second connection portion extending distally, the second connection portion having a second attachment surface facing and extending longitudinally aligned to the first attachment surface to define an overlapping portion and to define first and second seams extending longitudinally along first and second lateral extents of the overlapping portion; a first weld attaching the first and second connection portions, extending from the first seam toward the second seam (i.e. laterally); and a second weld attaching the first and second connection portions, extending from the second seam toward the first seam.

In an embodiment, the first (or proximal) connection portion can have different forms such as V-shape, U-shape, needle, needle with radiopaque marker, among others.

In an embodiment, a cross-sectional shape of the first connection portion perpendicular to the longitudinal axis is a section of an annulus. In another embodiment, a cross-sectional shape of the second connection portion perpendicular to the longitudinal axis is a circle.

In an embodiment, the first weld comprises a plurality of welding points along the first seam. In another embodiment, the second weld comprises a plurality of welding points along the second seam. In some embodiments, the plurality of welding points of the first and second welds are successive, i.e., the points are aligned.

In some embodiments, the first weld can further comprise a plurality of consecutive welding points. In some embodiments, the second weld can further comprise a plurality of consecutive welding points. Thus, a welding seam is provided on top of the welding points previously applied.

In an embodiment, the first weld and the second weld each extend along an entire length of the overlapping portion.

In an embodiment, a glue is applied over the first and second weld.

In an embodiment, a ratio of a cross-sectional area of the first connection portion perpendicular to the longitudinal axis to a corresponding cross-sectional area of the second connection portion perpendicular to the longitudinal axis is in a range of 1:4 to 2:1.

In an embodiment, a radius of the second attachment surface is smaller than a radius of the first attachment surface.

In an embodiment, the clot mobilizer device also includes a jacket extending around at least part of the overlapping portion. The jacket can extend proximally from the overlapping portion around at least part of the pusher.

Additionally, the clot mobilizer device can also include a radiopaque element disposed at a proximal end of the overlapping portion. In an embodiment, the radiopaque element comprises a coil extending around at least part of the pusher.

In an embodiment, the clot mobilizer device also comprises a jacket extending around the radiopaque element. The jacket can extend around at least part of the overlapping portion.

Embodiments of the present invention also provide, according to another aspect, a method for connecting an elongated device (e.g., a stent retriever or other medical device) to a pusher to enable the pusher to maneuver the elongated device (e.g. advance the elongated device distally, withdraw the elongated device proximally, and rotate the elongated device about a longitudinal axis from a proximal end of the pusher), the elongated device comprising a first connection portion (or a proximal connection portion) extending proximally, the first connection portion having a first attachment surface, the pusher comprising a second connection portion extending distally, the second connection portion having a second attachment surface. The method comprises disposing the first attachment surface longitudinally aligned to the second attachment surface to form an overlapping portion and to form first and second seams extending parallel to the longitudinal axis along first and second lateral extents of the overlapping portion; forming a first weld extending from the first seam toward the second seam, and forming a second weld extending from the second seam toward the first seam.

In an embodiment, a cross-sectional shape of the first connection portion perpendicular to the longitudinal axis is a section of an annulus. In another embodiment, a cross-sectional shape of the second connection portion perpendicular to the longitudinal axis is a circle.

In an embodiment, the step of forming the first weld comprises forming a plurality of welding points extending from the first seam toward the second seam. In another embodiment, the step of forming the second weld comprises forming a plurality of welding points extending from the second seam toward the first seam. Particularly, the first and second welds are formed by sequentially forming the plurality of welding points (i.e. the welding points are aligned).

In an embodiment, the step of forming the first weld further comprises consecutively forming a plurality of welding points extending from the first seam toward the second seam. In another embodiment, the step of forming the second weld further comprises consecutively forming a plurality of welding points extending from the second seam toward the first seam.

When energy, e.g. laser energy, is applied to form the first and second welds a heat affected zone appears (i.e. accumulation of heat in a specific zone) which can provoke a breakage of the first, the second, or both connection portions. To avoid that breakage, in some embodiments, the step of forming the first weld further comprises forming a first welding point of the plurality of welding points at a distal portion of the first seam and forming each other welding point of the plurality of welding points at a more proximal location along the first seam than a prior formed welding point of the plurality of welding points. In some embodiments, the step of forming the second weld further comprises forming a first welding point of the plurality of welding points at a distal portion of the second seam and forming each other welding point of the plurality of welding points at a more proximal location along the second seam than a prior formed welding point of the plurality of welding points.

In some embodiments, the first weld can extend along an entire length of the first seam and the second weld can extend along an entire length of the second seam.

In an embodiment, the step of forming a first weld comprises forming a first welding point at a proximal end of the first seam and a second welding point between the proximal end and the distal end of the first seam before forming a welding point at any other points along the first seam.

In an embodiment, the step of forming the second weld comprises forming a first welding point at a proximal end of the second seam and a second welding point between the proximal end and the distal end of the second seam before forming a welding point at any other points along the second seam.

In an embodiment, the step of forming the first weld comprises directing energy laterally at the first seam toward the second seam. In an embodiment, the step of forming the second weld comprises directing energy laterally at the second seam toward the first seam.

In an embodiment, the method also comprises the step of adding glue over the first and second welds.

In an embodiment, the method also comprises the step of placing a radiopaque element at a proximal end of the overlapping portion. In an embodiment, the radiopaque element can comprise a coil extending around at least part of the pusher.

In an embodiment, the method also comprises the step of assembling, or placing, the radiopaque element onto a distal end of the pusher and pushing the radiopaque element towards a proximal end of the pusher.

In an embodiment, the method further comprises covering at least part of the overlapping portion with a jacket. In some embodiments, a portion of the pusher proximal to the overlapping portion can also be covered with the jacket.

In an embodiment, the method further comprises covering the radiopaque element with a jacket. In some embodiments, the jacket also extends around at least part of the overlapping portion.

In an embodiment, before inserting the jacket, the method further comprises placing, or pushing back, the radiopaque element to the distal end of the pusher (or at the proximal end of the overlapping portion), without covering the overlapping portion.

In yet another embodiment, the method comprises covering at least part of the overlapping portion with a jacket (or inner jacket), the jacket extending proximally from the overlapping portion around at least part of the pusher; placing a radiopaque element at a proximal end of the overlapping portion, the radiopaque element comprising a coil extending around at least part of the pusher; and covering the radiopaque element with a jacket (or outer jacket), the jacket extending around at least part of the overlapping portion.

In an embodiment, before inserting the inner jacket, the method further comprises assembling, or placing, the radiopaque element onto a distal end of the pusher and pushing the radiopaque element towards a proximal end of the pusher.

In another embodiment, before inserting the outer jacket, the method further comprises placing, or pushing back, the radiopaque element to the distal end of the pusher (or at the proximal end of the overlapping portion), without covering the overlapping portion.

In an embodiment, a heat shrinkable material can be also added to the inner and outer jackets.

In an embodiment, a ratio of a cross-sectional area of the first connection portion perpendicular to the longitudinal axis to a corresponding cross-sectional area of the second connection portion perpendicular to the longitudinal axis is in a range of 1:4 to 2:1, particularly 3:4.

In an embodiment, a radius of the second attachment surface is smaller than a radius of the first attachment surface.

In some embodiments, according to the present invention, the clot mobilizer device also includes radiopaque markers located at a distal end of the working portion. In other embodiments the radiopaque markers can be also located at other sections of the working portion, for example at the middle and/or at the proximal end. The radiopaque markers particularly have different lengths to avoid entanglement between them or other devices. The radiopaque markers can be made of a Platinum Iridium alloy or of Tantalum.

In an embodiment, the clot mobilizer device is made of a metal including Nitinol. In particular, the Nitinol material complies with the ASTM (American Society of Testing and Materials) F2063 (Standard Specification for Wrought Nickel-Titanium Shape Memory Alloys for Medical Devices and Surgical Implants). Nitinol is well known for applications in self-expanding structures. However, other types of metals or even other types of materials can be also used, for example cobalt-chromium alloys or iron alloys such as stainless steel or spring steel; also, other materials with shape memory properties can be used, for example cooper or magnesium alloys.

In an embodiment, the clot mobilizer also includes an ultrasound transmission member/device to fragment the thrombus into smaller particles by applying ultrasound energy waves. In a particular embodiment, the ultrasound transmission member comprises a sonotrode operating at high frequency (between 15 and 30 kHz, particularly 19.8 kHz or 20 kHz). The vibrational amplitude of the sonotrode in this case is transmitted through the wire of the clot mobilizer. The vibrational amplitude can range between 3 μm (10%) and 7.5 μm (25%), particularly 6 μm (20%). The duration of the ultrasound transmission can be e.g. 60 seconds or 120 seconds, among others.

In an embodiment, the ultrasound transmission member particularly extends longitudinally through the axis of the clot mobilizer. Moreover, in some embodiments, a sonic connector can be also coupled to a proximal end of the ultrasound transmission member.

Another aspect of the invention provides a method of extracting a clot from a blood vessel of a patient by advancing a clot mobilizer device into the blood vessel in an unexpanded configuration having a compressed diameter of less than 1.5 mm, the clot mobilizer device comprising a plurality of crowns of cells in a working portion, each cell comprising an open area bordered by struts, a distal end of each cell in a first crown of the plurality of crowns being contiguous with a proximal end of a corresponding cell in a third crown of the plurality of crowns, a distal end of each cell in a second crown of the plurality of crowns being contiguous with a proximal end of a corresponding cell in a fourth crown of the plurality of crowns, the second crown between distal to the first crown and proximal to the third crown, the fourth crown being distal to the third crown, first and second opposite midportions of each cell in each of the first, second, third fourth crowns each being contiguous with a midportion of an adjacent cell in such crown; expanding the working portion of the clot mobilizer into the clot to an expanded diameter of at least 3.5 mm; and exerting an outward radial force with the working portion between 0.75 N and 3.0 N at every diameter between and including the compressed diameter and the expanded diameter. In some embodiments, the exerting step includes the step of exerting an outward radial force with the working portion between 0.75 N and 1.5 N when the expanded diameter is around 3.5 mm. In some embodiments, wherein the clot mobilizer device has a tapered portion extending proximally from the working portion, the method further includes the step of expanding the tapered portion to a diameter smaller than the expanded diameter of the working portion. In either or both of these embodiments, the advancing step may include the step of advancing a pusher extending proximally from a connection portion of the tapered portion outside of the patient.

In an embodiment, the aspiration funnel is self-expandable.

In an embodiment, the aspiration funnel comprises a segment defining a distal end and a proximal end, wherein the segment is formed by a mesh of at least two sets of helicoidal filaments turning respectively in opposite directions and being intertwined; the mesh comprises two distinct tubular sections, a first section and a second section, wherein the second section, adjacent to the first section, provides a reduction of diameter; and, said mesh of the first section has helicoidal filaments with a braiding angle configured to provide outward radial forces higher than in the second section, such that the first section becomes appositioned against the inner wall of the blood vessel.

In an embodiment, the first section of the mesh comprises closed loops at the distal end configured to act as a spring, such that the outward radial forces in a first and second end portions of the first section are higher than in an intermediate portion thereof.

In an embodiment, the second section of the mesh is comprised of two sub-sections, a first sub-section having a shape with a progressive reduction of diameter configured to open and create a space for the thrombus and to reduce (or stop) a proximal blood flow during the removal of the thrombus, and a second sub-section of a tubular uniform diameter configured to provide a connection to the aspiration catheter. In particularly, the shape of the first sub-section is cone-shaped (or funnel-shaped).

In an embodiment, the two sets of helicoidal filaments are adapted to become more longitudinally aligned as the aspiration funnel lengthens and narrows. In particularly, the helicoidal filaments of the mesh are made of a metal, a metal alloy or a composite including Nitinol or Nitinol/Platinum.

In a particular embodiment, the helicoidal filaments comprise a number ranging between 24 and 48, said filaments having a cross section comprised in a range between 40 and 60 μm; and the angle of the helicoidal filaments with regard to a longitudinal axis of the segment is comprised between 50 and 65 degrees for the first section, and between 15 and 50 for the second sub-section.

In a particular embodiment, the first section comprises a length ranging between 4 and 40 millimeters and the second sub-section comprises a length ranging between 1 and 10 millimeters; the first section comprises an outer diameter ranging between 3.5 and 6 millimeters and the second sub-section comprises an outer diameter ranging between 1 and 2 millimeters; and the shape of the first sub-section comprises a generatrix with an angle comprised between 15 and 45 degrees with regard to a longitudinal axis of the segment.

In a particular embodiment, the non-permeable covering of the aspiration funnel comprises a polymer including silicone or polyurethane.

### BRIEF DESCRIPTION OF THE DRAWINGS

The previous and other advantages and features will be more fully understood from the following detailed description of embodiments, with reference to the attached figures, which must be considered in an illustrative and non-limiting manner, in which:

FIGS. 1A-1F illustrate different examples of the proposed clot mobilizer device for extraction of an occlusion from a blood vessel.

FIGS. 2A and 2B illustrate enlarged views of the tapered portion of the clot mobilizer device, according to some embodiments of the present invention.

FIGS. 3A and 3B illustrate other embodiments of the clot mobilizer device adapted to be advanced distally and withdrawn proximally from a proximal end and rotated about a longitudinal axis from the proximal end.

FIGS. 4A-4D illustrate different designs of the connection portion. FIG. 4A: V-shape; FIG. 4B: U-shape; FIG. 4C: needle shape; and FIG. 4D: needle with radiopaque marker.

FIG. 5 schematically illustrates the geometry of the cross-sectional view of the first connection portion and the second connection portion of the clot mobilizer device, according to an embodiment.

FIG. 6 illustrates a more detailed view of a clot mobilizer device adapted to be advanced distally and withdrawn proximally from a proximal end and rotated about a longitudinal axis from the proximal end, according to another embodiment.

FIG. 7 is a flow chart illustrating a method for connecting an elongated device to a pusher to enable the pusher to maneuver the elongated device, according to an embodiment.

FIG. 8 graphically illustrates a comparison (individual radial force vs. diameter) between the proposed clot mobilizer device and the Solitaire device.

### DETAILED DESCRIPTION OF PARTICULAR EMBODIMENTS

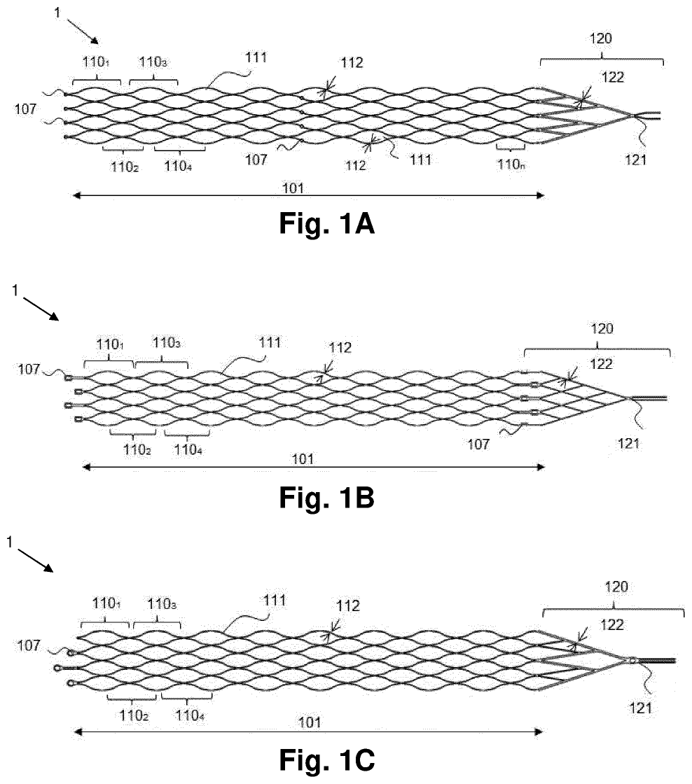

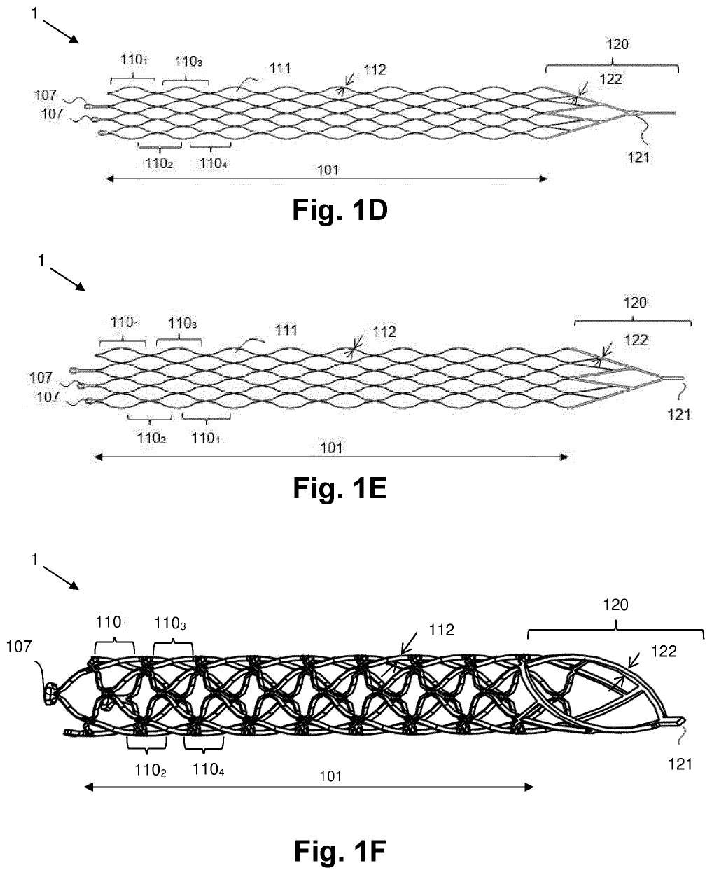

With reference to FIGS. 1A-1F, therein different embodiments of the proposed clot mobilizer device 1 are illustrated. In any of these embodiments, the clot mobilizer device 1 includes a working portion 101 comprising a plurality of crowns 110₁ . . . 110ₙ of almond-shaped cells 111 defining a tubular-shaped section forming a cylindrically closed structure (as can be seen in the 3D representation illustrated in the embodiment of FIG. 1F). Moreover, the clot mobilizer device 1 also includes a tapered portion 120 that extends proximally from a proximal end of the working portion 101. For purposes of this disclosure, a “crown” is a closed ring extending circumferentially around the device with alternating longitudinally longer and longitudinally shorter sections. The working portion 101 in a compressed diameter can have a length between 10 mm and 60 mm, and the working portion 101 in an expanded diameter can have a length between 20 mm and 50 mm.

A distal end of each cell 111 in a first crown 110₁ is contiguous with a proximal end of a corresponding cell 111 in a third crown 110₃, and a distal end of each cell 111 in a second crown 110₂ is contiguous with a proximal end of a corresponding cell 111 in a fourth crown 110₄. The midportions of each cell 111 in each of the crowns each are also contiguous with a midportion of an adjacent cell 111 in such crown.

The clot mobilizer device 1 can be made of Nitinol, cobalt-chromium alloys, iron alloys such as stainless steel or spring steel, etc.

The clot mobilizer device 1 illustrated in FIGS. 1A-1F also include radiopaque markers 107. The radiopaque markers 107 can be incorporated at a distal end of the working portion 101 only (as in FIGS. 1C-1F) or can be also incorporated at other sections of the working portion 101 (as in FIGS. 1A and 1B). In some embodiments, the different radiopaque markers 107 have different lengths to avoid entanglement between them or other devices. The radiopaque markers 107 can be made of a Platinum Iridium alloy or of Tantalum.

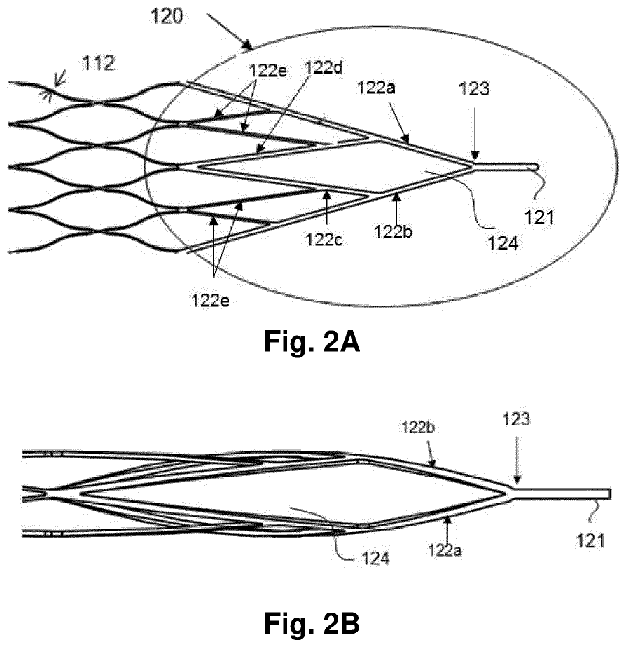

Each one of cells 111 has struts 112. Likewise, the tapered portion 120 also has at least some wide struts 122. Particularly, the wide struts 122 of the tapered portion 120 have a width greater than a width of the working portion struts 112 (see FIGS. 2A and 2B for an enlarged illustration thereof).

For example, in the embodiment shown in FIGS. 2A and 2B, first 122a and second 122b struts, extending proximally from the proximal end of the working portion 101, converge at a distal end 123 of a proximal (or a first) connection portion 121 of the clot mobilizer device 1. Third 122c and fourth 122d struts extend distally from first 122a and second 122b struts, respectively, and converge at the proximal end of the working portion 101. First 122a, second 122b, third 122c and fourth 122d struts together define a cell 124 in the tapered portion 120, and these struts 122a-122d are all thicker than the struts 112 in the working portion 101. In the embodiment illustrated in FIGS. 2A and 2B, first, second, third and fourth struts 122a-122d are thicker than other struts 122e in the tapered portion 120. These thicker struts 122a-122d help provide greater outward radial force to the working portion 101 from its compressed diameter to its expanded diameter, as discussed below. In some embodiments, because the working portion 101 and the tapered portion 120 of the clot mobilizer device 1 is formed from a single hypotube, the wide tapered portion struts 122 have a thickness equal to the thickness of the working portion struts 112, which is predetermined by the thickness of the tube from which it is made. In some embodiments, the working portion struts 112 can have a width between 30 μm and 60 μm, and at least some of the tapered portion struts 122 can have a width between 60 μm and 155 μm.

In some embodiments, the working portion 101 has a compressed diameter of less than 1.5 mm and an expanded diameter of approximately 3.5 mm. The working portion 101 can exert an outward radial force between 0.75 N and 3 N at every diameter between and including the compressed diameter and the expanded diameter. In a particular embodiment, the outward radial force exerted by the working portion 101 in a compressed diameter of 1.5 mm is between 1.75 N and 3 N, more particularly 2.1 N; and the outward radial force exerted by the working portion 101 in an expanded diameter of 3.5 mm is between 0.75 N and 1.5 N, more particularly 1 N.

In order to calculate the outward radial force, in an embodiment, e.g. a time-diameter variation can be used. To that end, the clot mobilizer device 1 is introduced in a 12 segments head (in particular a RX550 from Machine Solutions Inc. as further detailed in the below examples) that allows compressing the device uniformly, with very low friction force. The temperature is set to 37±2° C. and the radial force bench decreases and increases the diameter according to specified profile, recording a radial force vs diameter curve.

Table 1 indicates some of the main specifications of the clot mobilizer device 1 illustrated in FIGS. 1A-1E.

TABLE 1
Clot mobilizer device specifications. distal = distal
section of the working portion; middle = middle
section of the working portion; proximal = proximal
section of the working portion; tapered = tapered portion.

| | Radiopaque | | | |
|---|---|---|---|---|
| | Marker | Radiopaque | Tapered | |
| | Quantity and | Marker | Section | Attachment |
| Prototype | Location | Form | Design | Design |
| ID19 | 4 distal + 4 | Circular | Uniform | V-shape |
| (FIG. 1A) | middle + 4 tapered | shaped | | |
| ID20 | 4 distal + 4 | Rectangular | Diamond | U-shape |
| (FIG. 1B) | tapered | shaped | | |
| ID25 | 3 distal + 1 | Oval | Uniform | U-shape |
| (FIG. 1C) | proximal | shaped | | |
| ID34 | 3 distal + 1 | Oval | Uniform | Needle |
| (FIG. 1D) | proximal | shaped | | |
| ID45 | 3 distal | Slotted | Uniform | Needle |
| (FIG. 1E) | | shaped | | |

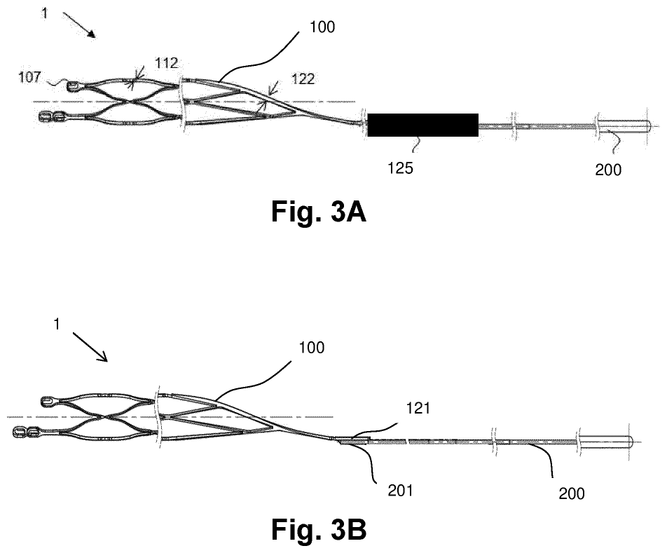

FIGS. 3A and 3B illustrate a clot mobilizer device 1 according to some embodiments of the present invention. According to these embodiments, the proposed clot mobilizer device 1 comprises an elongated device 100, which is formed by the cited working element 101 and tapered section 120 and is attached to a pusher 200, forming an attachment (assemblage) 125, see FIG. 3A. As shown in FIG. 3B, the proposed clot mobilizer device 1 comprises the elongated device 100 and the pusher 200, where the elongated device 100 is attached to the pusher 200 via the first (or proximal) connection portion 121 and a second connection portion 201.

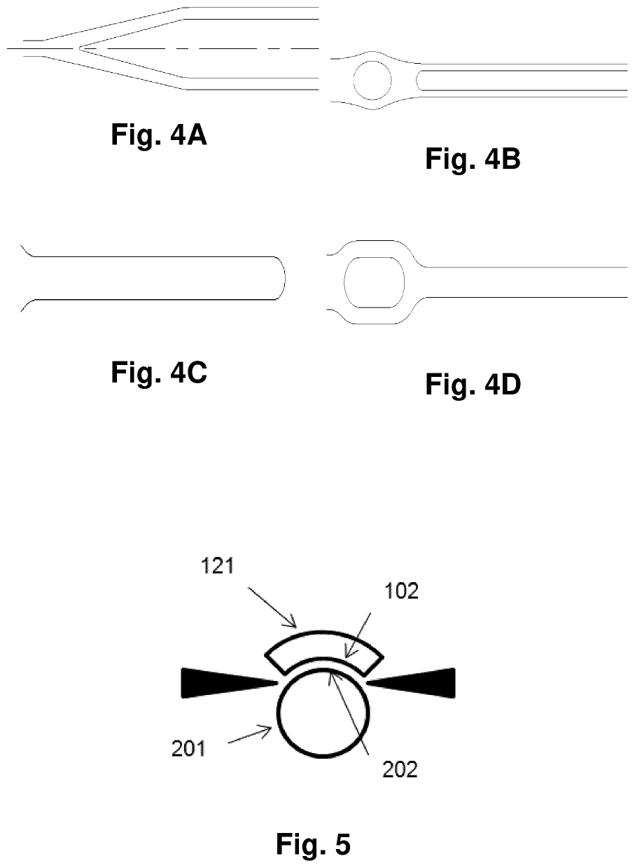

FIGS. 4A-4D illustrate different types of connection portions 121. The elongated device 100 can comprise different medical devices, particularly a stent retriever, and includes the first connection portion 121 extending proximally, the first connection portion 121 having a first attachment surface 102 (see FIG. 5). The pusher 200 can comprise a second connection portion 201 extending distally, the second connection portion 201 having a second attachment surface 202 facing and extending longitudinally aligned to the first attachment surface 102 to define an overlapping portion and to define first and second seams extending longitudinally along first and second lateral extents of the overlapping portion.

The clot mobilizer device 1 also comprises a first weld attaching the first and second connection portions 121, 201, extending from the first seam toward the second seam, and a second weld attaching the first and second connection portions 121, 201, extending from the second seam toward the first seam.

FIG. 5 illustrates an embodiment of the cross-sectional shape of the first connection portion 121 being a section of an annulus and of the cross-section shape of the second connection portion 201 being a circle. The black triangles show the direction of how the energy, particularly laser energy, is laterally applied to form the first and second welds. In this embodiment, the radius of the second attachment surface 202 is smaller than the radius of the first attachment surface 102.

Particularly, the ratio of the cross-sectional area of the first connection portion 121 perpendicular to the longitudinal axis to the corresponding cross-sectional area of the second connection portion 201 perpendicular to the longitudinal axis is in the range of 1:4 to 2:1; more particularly the ratio is 3:4.

The first weld can comprise multiple welding points along the first seam. Likewise, the second weld can also comprise multiple welding points along the second seam. In an embodiment, the welding points comprise a number between 2 and 12 welding points. In another embodiment, the welding points comprise a number between 2 and 5. The welding points are particularly aligned with each other. In some embodiments, the first weld and the second weld each can additionally comprise a welding seam disposed/arranged over the multiple welding points previously applied and formed by a plurality of consecutive welding points.

In some embodiments, the first weld and the second weld each extend along an entire length of the overlapping portion.

In some embodiments, glue can be added over the first and second welds.

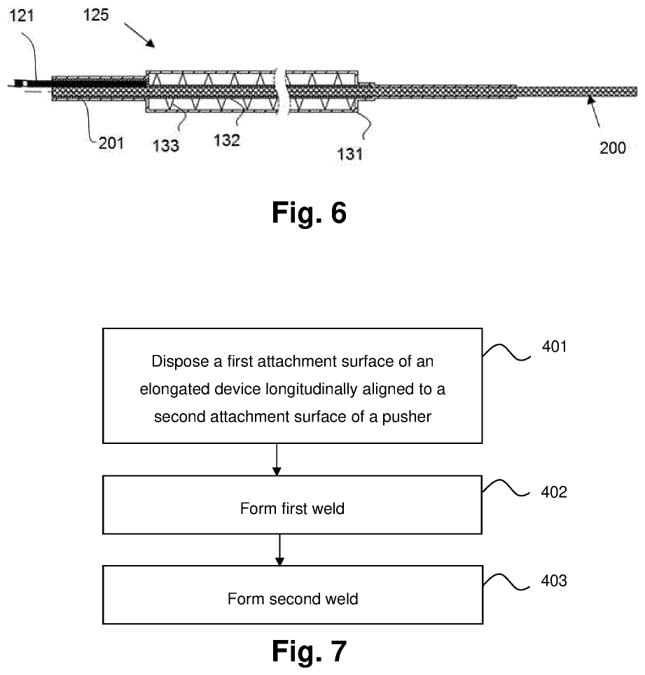

FIG. 6 illustrates a particular embodiment of how the elongated device 100 connects to the pusher 200 by means of the attachment 125. According to this particular embodiment, the clot mobilizer 1 can comprise a jacket 131 or outer jacket, a jacket 132 or inner jacket, and a radiopaque coil 133 as a radiopaque element extending around the pusher 200, wherein the radiopaque coil 133 is arranged between the outer and inner jackets 131, 132 respectively. The jacket 132 extends proximally from the overlapping portion over a portion of the pusher 200. The jacket 131 extends around the radiopaque coil 133 and part of the overlapping portion. Additionally, a heat shrinkable material can be added to the inner jacket 131 and outer jacket 132.

FIG. 7 illustrates a method for associating (i.e. connecting/assembling) the elongated device 100 to the pusher 200 to enable the pusher 200 to maneuver the elongated device 100 (i.e. advance the elongated device 100 distally, withdraw the elongated device 100 proximally, and rotate the elongated device 100 about a longitudinal axis from a proximal end of the pusher 200). At step 401 the first attachment surface 102 of the first connection portion 121 is disposed longitudinally aligned to the second attachment surface 202 of the second connection portion 201 to form an overlapping portion and to form first and second seams extending parallel to the longitudinal axis along first and second lateral extents of the overlapping portion. At step 402 a first weld extending from the first seam towards the second seam is formed. At step 403 a second weld extending from the second seam toward the first seam is formed.

In some embodiments, step 402 comprises forming or applying a plurality of welding points (for example between 2 and 12, particularly between 2 and 5) extending from the first seam toward the second seam. Likewise, step 403 comprises forming or applying a plurality of welding points extending from the second seam toward the first seam. In some embodiments, the plurality of welding points are aligned with each other. In some embodiments, steps 402 and 403 also comprise forming a welding seam by consecutively forming or applying a plurality of welding points on top of the previously formed/applied welding points.

To avoid accumulation of heat in specific zones, in some embodiments step 402 further comprises forming a first welding point of the plurality of welding points at a distal portion of the first seam and forming each other welding point of the plurality of welding points at a more proximal location along the first seam than a prior formed welding point of the plurality of welding points, and step 403 further comprises forming a first welding point of the plurality of welding points at a distal portion of the second seam and forming each other welding point of the plurality of welding points at a more proximal location along the second seam than a prior formed welding point of the plurality of welding points. That is, the energy device forming the weld is moved from the distal end of the first (and second) seam to the proximal end of said seam in order to avoid that the first connection portion 121, the second connection portion 102, or both, accumulate the heat which could provoke a breakage thereof.

In certain embodiments, the method includes covering a portion of the pusher 200 proximal to the overlapping portion with a jacket 132 (or inner jacket); placing a radiopaque element 133 at a proximal end of the overlapping portion and extending the radiopaque element 133 around the pusher 200; and covering the radiopaque element 133 with a jacket 131 (or outer jacket) and extending the later at least part of the overlapping portion.

In an embodiment, before inserting the inner jacket 132, the method can further comprise assembling, or placing, the radiopaque element 133 onto a distal end of the pusher 200 and pushing the radiopaque element 133 towards a proximal end of the pusher 200.

In another embodiment, before inserting the outer jacket 131, the method can further comprise placing, or pushing back, the radiopaque element 133 to the distal end of the pusher 200 (or at the proximal end of the overlapping portion), without covering the overlapping portion.

In some embodiments, before inserting the outer jacket 131, the method can further comprise heat shrinking the inner jacket 132 onto the overlapping portion, and after the outer jacket 131 has been inserted, heat shrinking the outer jacket 131 onto the inner jacket 132 and the radiopaque coil 133. The heat shrinking can be made using a suitable shrinkable material, for example a thermoplastic material such as polytetrafluoroethylene (PTFE); fluorinated ethylene propylene (FEP); perfluoroalkoxy alkanes (PFA); ethylene tetrafluoroethylene (ETFE); polyethylene terephthalate (PET) resins, etc.

In other embodiments, the elongated device 100 can be associated (i.e. connected/assembled) to the pusher 200 by other suitable attachment techniques, such as chemical or thermally bonding, friction or mechanically fitting, crimping, soldering, brazing, or even by using a connector material or member, or combinations thereof. In another embodiment, the elongated device 100 can be associated to the pusher 200 by crimping a coated or plated band disposed around and crimped to the overlapping portion.

Following, different examples of the use and performance of the proposed clot mobilizer device 1 are detailed. The examples and drawings are provided herein for illustrative purposes, and without intending to be limiting to the present invention.

### Example 1: In Vitro 3D Simulation Model, Pre-Clinical Data

### INTRODUCTION

Endovascular treatment (EVT) is recognized as the most effective treatment for large vessel occlusion (LVO) strokes. Highest degree of recanalization in the shortest time with the minimum number of attempts has been demonstrated to correlate with improved clinical outcomes. Although highly effective, failure to reach complete recanalization has been reported in about 20% of treated patients. In order to improve patient outcomes, different devices and combinations are under development to increase the first pass complete recanalization rate. The development of such devices includes preclinical testing in phantom models simulating the cerebrovascular human anatomy, and animal models in which device related vessel injury can be assessed. Each simulation model has its own characteristics and therefore it is recommended that any new device or combination will prove its efficacy and safety in different conditions before final evaluation in a first in human study.

The clot mobilizer device 1 (hereinafter Conda) is a stent-like device, with the capacity of recanalize an occlusion from a blood vessel. In these embodiments, particularly, Conda is formed mainly by a stent and a pusher wire. The prototype used in this first example is the ID45 (FIG. 1E) and it presents oval shaped cells and a tapered section that joints the stent with the pusher wire through a needle-shape attachment.

This study was aimed to assess the efficacy and performance of the Conda prototype in comparison with Solitaire™ (4×40 mm, Medtronic, hereinafter Solitaire), the currently most used commercial stent retriever, in an in vitro 3D simulation model, a cerebrovascular model of the intracranial circulation that simulates the carotid and cerebral physiological blood flow, pressure and vessel anatomy including an occlusive ex vivo clot analog. Solitaire is designed for use in the flow restoration of patients with ischemic stroke due to large intracranial vessel occlusion. Solitaire is a stent-like device, which can be fully deployed, fully resheated, and recovered. It is available in 2 sizes: 4 and 6 mm in diameter.

In order to mimic the clinical scenario, the Clot Mobilizers (CM), Conda and Solitaire, were used in this study in combination with other devices: the ANA Aspiration Catheter device (from Anaconda Biomed, hereinafter ANA), which is comprised of two coaxial catheters: the delivery catheter (comprises a hydrophilic coating to reduce friction and radiopaque markers on the distal end) and the aspiration catheter (comprised by a catheter with a funnel shape in the distal end, able to provide local flow restriction); and the 8Fr FlowGate2™ Balloon Guide Catheter 95 cm (from Stryker Neurovascular, hereinafter BGC), which is a balloon guide catheter able to offer proximal flow control and stable platform to facilitate the insertion and guidance of an intravascular catheter. However, the objective of the study is to compare the performance and efficacy of Conda and Solitaire.

The main objectives of the study were:
Assessment of the efficacy of Conda and Solitaire in combination with ANA or BGC in terms of the rate of revascularization and rate of clot embolization.
Assessment of the performance of Conda and Solitaire in combination with ANA or BGC to reach the target vessel, to deploy the stent, and to withdrawal.

Methods

The tests were carried out in the Animal Facility of the Vail d'Hebrón Institut de Recerca (VHIR), Barcelona (Spain).

Mechanical thrombectomies with ANA and BGC, in combination with both CMs (Conda and Solitaire), were simulated in the model cerebrovascular occlusion (including a clot analog). The procedures were followed by a low-resolution fluoroscopy and assisted by trained technicians.

The cerebrovascular model system was composed of a human vascular replica and a physiologically relevant mock circulation flow loop. A three-dimensional in vitro model of the intracranial circulation was used as vascular replica.

Two 3D models were used to simulate the mechanical thrombectomy interventions:
Vascular model Jacobs Institute V3.4 (JI V3.4): This was designed based on patient vascular anatomy using CT-A imaging (50 patients) and then printed on an 3D printer (Jacobs Institute). The model closely resembles the human intracranial circulation in terms of curvature, diameter, and length, and consists on the internal carotid artery segment and middle cerebral artery branches (M1-M4 segments), bilateral A1 anterior cerebral artery segments connected to a single anterior cerebral artery, and a single posterior communicating artery, thus allowing near complete circle of Willis circulation (intracranial model).
Vascular model UMASS: This model was manufactured by 3D printing using transparent silicone and it presents a hydrophilic coating on its inner surface to make it more slippery. It is based on clinical data, and is a transparent model, so it does not require fluoroscopy for its usage. The UMASS in vitro model mimics the geometry of the intracranial zone of a patient; it does not include the femoral access, the Aortic arch or the complete pathway of the Carotid artery. It only contains the last curve of the access zone. It consists in two challenging levels available for thrombectomy, one of them more severe than the other.

For the assessment of the efficacy in clot retrieval (revascularization and embolization rates), soft red and fibrin rich clots were used to generate middle cerebral artery (MCA; M1 & M2) occlusions.
Soft red clot (5×8 mm): 4 ml of non-anticoagulated porcine blood was mixed with 32 mg of fibrinogen from bovine plasma (F8630, Sigma-Aldrich) and 1 unit of thrombin form bovine plasma (T4648, Sigma-Aldrich) for at least 3 min. The mixture was incubated at room temperature for at least 60 min.
Fibrin rich clot (8×8×8 mm): Porcine blood was anticoagulated using sodium citrate solution (3.2%) immediately after collection. The whole blood constituents were subsequently separated using centrifugation (600 g, 15 min, 4° C.) and the extracted plasma was mixed with the red blood cells (RBCs) in a ratio of 9:1. Coagulation was initiated by the addition of calcium chloride (2.06%) and the clotted material was allowed to mature for 60 min at 37° C. The resultant clots consist of approximately 100% fibrin.

The clots were injected into the flow loop to form an MCA occlusion. Prior to initiating thrombectomy, complete occlusion with TICI 0 was required. TICI scale is the standard score to assess the revascularization rate and is defined as follows:
TICI scoring grade 0: no perfusion.
TICI scoring grade 1: antegrade reperfusion past the initial occlusion but limited distal branch filling with little or slow distal reperfusion.
TICI scoring grade 2a: antegrade reperfusion of less than half of the occluded target artery previously ischemic territory (e.g. in one major division of the middle cerebral artery (MCA) and its territory).
TICI scoring grade 2b: antegrade reperfusion of more than half of the previously occluded target artery ischemic territory (e.g. in two major divisions of the MCA and their territories).
TICI scoring grade 3: complete antegrade reperfusion of the previously occluded target artery ischemic territory, with absence of visualized occlusion in all distal branches.

Other used guidance/support devices in combination with ANA or BGC, Conda and Solitaire are shown in Table 2:

TABLE 2
Support devices used in combination with ANA,
BGC, Conda and Solitaire during interventions.

| Device type | | Name | Company |
|---|---|---|---|
| Neurovascular | Guide catheter | Neuronmax 088 | Penumbra |
| guide | Microcatheter | Rebar 18 Micro Catheter | Covidien |
| catheters | | Marksman Catheter | Medtronic |

Procedure of Mechanical Thrombectomy

Neuron Max 088 guide catheter was placed in the cervical Internal Carotid Artery (ICA) and delivered the guidewire which was then softly advanced through the target vessel.

The ANA device was combined with both CMs independently to retrieve the clot: with the stented funnel deployed proximal to the occlusion, the microcatheter with the CM in it was navigated through the aspiration catheter and the deployed stented funnel over the wire until reaching and crossing the clot. The CM was deployed to capture the clot while continuous aspiration was applied via ANA (aspiration was applied after the microcatheter was retrieved), the CM was dragged until the whole clot was safety placed inside the stented funnel and both devices were finally withdrawn as one. When using BGC device, it was combined with both CM independently to retrieve the clot, following the standard procedure described in the instructions for use of BGC.

Flow and revascularization rates were evaluated to assess the efficacy following the procedure time points after all procedure execution: pre-clot placement (for baseline), pre-treatment (clinical starting point) and post-thrombectomy (first, second and third passes of revascularization). TICI 2b and 3 are considered successful revascularization (1). TICI 0, 1, and 2a are considered unsuccessful revascularization (0). The main endpoints considered in the efficacy assessment were: Rates of revascularization after first and third passes (TICI 2b-3).

Also, Distal Territory (EDT) and Emboli New Territory (ENT) were assessed. EDT score of 0 and ENT score of 0 is indicative of no embolic events. EDT score of 1 and ENT score of 1 is indicative of an embolic event. The procedure time points after all procedure execution were followed, and the main endpoints considered in the efficacy assessment were EDT and ENT after first and third passes (TICI 2b-3).

The performance of Conda, in the model JI V3.4, was assessed after the first attempt by studying the following endpoints depending on their quantification (i.e. score):
IS-COMP: Conda Introducer Sheath (IS) compatibility with the Hemostatic Valve and Luer of the funnel; [1=poor behavior, CM could be damaged during introduction; 2=usual behavior compared to other brands; 3=easy to introduce into the HYV and through the luer but gets into the CM; 4=easy to introduce into the HYV and through the luer, does not get into the CM].
C-PUSH: Pushability of the Conda to the target vessel. Access from the IS to the microcatheter; [1=poor; 2=moderate-adequate; 3=very good].
S-TARGET: Conda reaches the target zone; [1=poor; 2=moderate-adequate; 3=very good].
S-ACCURACY: Accuracy of the deployment of the Conda in the target vessel; [1=poor accuracy, target vessel not reached; 2=moderate accuracy target vessel not reached; 3=moderate accuracy target vessel reached; 4=very accurate, deployed in the target vessel].
S-ADAPTABILITY: Conda adaptability (with the clot and target vessel); [1=stent does not expand; 2=stent self-expands but not completely; 3=stent self-expands integrating the clot but not completely adapts to the vessel; 4=stent self-expands integrating the clot and adapting to the vessel].
C-VISIBILITY: Visibility of the Conda under fluoroscopy; [1=poor; 2=moderate-adequate; 3=very good].
S-F-ENTANGLEMENT: Compatibility of the funnel and the Conda; [1=entanglement; 2=few interactions; 3=no interaction].
S-RETRIEVAL: Resistance of the Conda during retrieval; [1=friction during withdrawal; 2=easy withdrawal].

Finally, a visual inspection to quantify the integrity of the Conda and the funnel of the aspiration catheter was performed after all procedure executions.

Results and Conclusions

The following Table 3 presents the results of the efficacy obtained comparing the use of Conda and Solitaire when ANA device is used:

TABLE 3
Results of efficacy of Conda and Solitaire in conjunction with ANA.

| | | | ANA + Conda | | | ANA + Solitaire | | |
|---|---|---|---|---|---|---|---|---|
| CEREBROVASCULAR MODEL | | CLOT | | 1^{st} | 3^{rd} | | 1^{st} | 3^{rd} |
| Model | Tortuosity Index | TYPE | n | PASS | PASS | n | PASS | PASS |
| JACOBS | access: 5.464 | SOFT | 12 | 100% | 100% | 10 | 90% | 90% |
| V3.4 | intracerebral: 3.859 | RED | | | | | | |
| | global: 9.323 | FIBRIN | 15 | 100% | 100% | 15 | 67% | 73% |
| | | RICH | | | | | | |
| UMASS | access: 0.047 | SOFT | 10 | 100% | 100% | 10 | 100% | 100% |
| MODERATE | intracerebral: 4.273 | RED | | | | | | |
| | global: 4.320 | FIBRIN | 15 | 93% | 100% | 15 | 80% | 93% |
| | | RICH | | | | | | |

The results obtained with the Conda device were superior to the ones of the Solitaire in all the different testing combinations (Model and Clot type) indicating a better efficacy of the Conda device in thrombectomy procedures. The highest differences were observed in the extraction of fibrin rich simulated thrombi. This type of thrombi is harder, and it has a higher tendency to be rolled-out from the stent during retrieval.

Table 4 presents the results of efficacy obtained comparing the use of Conda and Solitaire when BGC device is used:

TABLE 4
Results of efficacy of Conda and Solitaire in conjunction with BGC.

| | | | BGC + Conda | | | BGC + Solitaire | | |
|---|---|---|---|---|---|---|---|---|
| CEREBROVASCULAR MODEL | | CLOT | | 1^{st} | 3^{rd} | | 1^{st} | 3^{rd} |
| Model | Tortuosity Index | TYPE | n | PASS | PASS | n | PASS | PASS |
| JACOBS | access: 5.464 | SOFT | 10 | 90% | 90% | 10 | 100% | 100% |
| V3.4 | intracerebral: 3.859 | RED | | | | | | |
| | global: 9.323 | FIBRIN | 10 | 93% | 100% | 15 | 40% | 67% |
| | | RICH | | | | | | |
| UMASS | access: 0.047 | SOFT | 10 | 100% | 100% | 10 | 100% | 100% |
| MODERATE | intracerebral: 4.273 | RED | | | | | | |
| | global: 4.320 | FIBRIN | 15 | 80% | 87% | 15 | 93% | 100% |
| | | RICH | | | | | | |

In general, the results obtained using BGC were worse than with ANA device due to the larger path that the thrombi had to travel from the M1 of the cerebrovascular model to the tip of the BGC in which the aspiration is applied. Conda showed more consistent results obtaining more than 80% of success in 1^{st} pass in all the possible situations evaluated. In the Jacobs model with fibrin rich clot the Solitaire intervention obtained an efficacy equal to 40% and 67% at 1^{st} and 3^{rd} pass, respectively; on the other hand, Conda obtained an efficacy equal to 93% and 100% in 1^{st} and 3^{rd} pass, respectively.

The efficacy obtained with the Conda device was higher than with the Solitaire when used with ANA device. When used with BGC the results obtained with Conda device was lower than with ANA device but there are maintained in a similar level as Solitaire. These results imply that the Conda is an effective device for the extraction of clots, and it is the better choice than Solitaire when used together with the ANA device.

In the following Table 5 the results of performance obtained with the different interventions using Conda are presented; for example, the endpoint of pushability (C-PUSH) obtained a score of 3 in 10 interventions (100% of all interventions).

TABLE 5
Performance endpoints scores of Conda after
the first pass using the JI V3.4 model.

| | % of interventions | | | | |
|---|---|---|---|---|---|
| | per score (number | | | | |
| | of interventions) | | | | Mean/Max. |
| Endpoint | 1 | 2 | 3 | 4 | Score |
| IS-COMP | 0% | 0% | 0% | 100% | 4/4 |
| | | | | (10) | |
| C-PUSH | 0% | 0% | 100% | — | 3/3 |
| | | | (10) | | |
| S-TARGET | 0% | 0% | 100% | — | 3/3 |
| | | | (10) | | |
| S-ACCURACY | 0% | 0% | 0% | 100% | 4/4 |
| | | | | (10) | |
| S-ADAPATABILITY | 0% | 0% | 0% | 100% | 4/4 |
| | | | | (10) | |
| C-VISIBILITY | 0% | 100% | 0% | — | 2/3 |
| | | (10) | | | |
| S-F-ENTANGLEMENT | 0% | 0% | 100% | — | 2/2 |
| | | | (10) | | |
| S-RETRIEVAL | 0% | 100% | — | — | 1/1 |
| | | (10) | | | |

All the end points evaluated showed that the Conda device has a very good behavior in reaching the target vessel, being adapted to the vessel wall, and without problems during the retrieval. The only endpoint in which was not possible to obtain the best qualification was in the visibility of the device under fluoroscopy. However, the visibility level obtained was considered acceptable but sub-optimal.

Finally, the compatibility of Conda device with ANA was studied by quantifying the integrity of the CM. As a result, no damage was found in the different Conda devices due to their use in conjunction with ANA device. It is considered that Conda device can be used with ANA without risk.

This study showed that Conda in conjunction with ANA obtained better results than Solitaire in conjunction with ANA. On the other hand, Conda showed similar results to Solitaire when they were used in conjunction with BGC. These results imply that the Conda is an effective device for the extraction of clots, and it is the better choice than Solitaire when used together with the ANA device. Conda also showed very good behavior in reaching the target vessel, being adapted to the vessel wall, and without problems during the retrieval. Finally, no damage was found in the Conda device due to their use in conjunction with ANA device, confirming that is totally compatible with ANA.

As a final conclusion, Conda is a clear option to be used in clinical trials to increase the revascularization rate during mechanical thrombectomies.

### Example 2: In Vivo Study of the Conda Clot Mobilizer Prototypes: Assessment of the Safety and Performance in the Porcine Model of Thrombectomy

### Introduction

Different prototypes of the Conda were analyzed in this study. In these embodiments, particularly, Conda is formed mainly by a stent and a pusher wire. Different prototypes were assessed and characterized in different in vitro and in vivo studies in order to improve the design characteristics.

This study aimed to assess the safety and performance of the Conda prototypes in the in vivo model of vessel injury and recanalization in porcine arteries, in order to test the vascular damage produced by the device to the vascular tissue and its performance reaching a target site on blood vessels of an animal swine model, and compare the clot mobilizer device with a similar marketed stent retriever (Solitaire™ revascularization device, manufactured by Medtronic, hereinafter as Solitaire), as shown in Example 1.

This experimental study also aimed the evaluation of some aspects of the clot mobilizer performance and usability, including the device maneuverability in clot retrieval, although, this animal model is limited to assess navigability through highly tortuous complex vascular anatomy (such as in the human cerebral vasculature).

In order to mimic the clinical scenario, the Clot Mobilizers (CM), Conda and Solitaire, were used in this study in combination with a microcatheter and with ANA (already described in Example 1) or a standard guide catheter, to reach the target vessel.

This study was aimed to assess the following:
The safety; any vascular injury at the renal arteries, triggered by the Conda prototypes in comparison with the marketed device Solitaire, both in combination with ANA or standard guide catheter, both systems after 3 passes.
The performance (effectiveness) and usability of Conda prototypes, in comparison with Solitaire, in combination with ANA or standard guide catheter, when used under a simulated procedure and environment, with or without a clot occlusion in the vessel lumen.
The radial force mechanical value that characterizes the force that the complete length of the stent will exert against the wall of the blood vessel.

Methods

This study was performed in the Animal Facility of the Vall d'Hebrón Institut de Recerca (VHIR), Barcelona (Spain).

16 female swines with a weight between 30 and 35 Kg of the species Sus scrofa were used as experimental subjects. All of them without diseases and under healthy conditions and facilitated by A. M. Animalia Bianya S. L. (from Girona, Spain). Animals were numbered as: C21, C22, C23, C25, C27, C28, C29, C30, C31, C33, C34, C35, C36, C37, C38 and C39.

The renal arteries of the swines were treated with different devices and the distal branch of each renal artery that was not treated was used as negative control. In each animal, after renal artery interventions had been completed, a synthetized radiopaque thrombus was inserted in the subclavian artery or a branch of the carotid artery to generate an intentional vascular blockade, followed by a recanalization attempt to retrieve the clot with the CMs.

The porcine model is a model already established for the evaluation of neurovascular and intravascular devices and techniques. This model is also useful to assess mechanical thrombectomy systems indicated for patients with large ischemic stroke. This model mainly allows assessing the vascular damage exerted by the device during use (security), and it also provides information on the performance and usability of the device in an in vivo model (efficacy) including the thrombectomy approach, in arteries of similar diameter to the cerebral arteries, which provide information further on the adequacy of its design. These models are very good for assessing the usability of the device: navigability, maneuverability and ability to access the target vessel, the efficacy in the thrombus capture and retrieval, the assessment of thrombus fragmentation and embolization.

The clot mobilizers used in this study were designed for use in the neurovasculature, such as the internal carotid artery, M1 and M2 segments of the MCA, anterior cerebral artery, and the basilar and vertebral arteries. Clot mobilizers used in this study are defined in Table 1 and/or FIGS. 1A-1E, which are the designs with similar structure than the frozen design. Other prototypes were evaluated but they are not shown in the current experimental example due to their difference in structure in comparison with the frozen design developed by Anaconda Biomed.

Procedure

The intervention was performed in a surgical room equipped with a fluoroscopy, echography, anesthesia and monitoring equipment. The general management of the intervention was performed by the facility veterinarian staff, and the specific procedure with the neurothrombectomy devices was assisted by a specialized interventionalist. The intervention with the thrombectomy devices was followed by fluoroscopy.

Each animal procedure started with a vascular injury performance assessment, that consisted in the intervention to the cranial or caudal branch of the right/left renal arteries and performance of a device deployment without clot removal in the chosen branch, while the adjacent branch (cranial or caudal) of the same side in the renal circulatory system artery (not subjected to any intervention) was used as a negative control artery.

Each intervention (after ensuring access of the device to the target section of the artery) consisted of three repetitions deployment of the tested devices (CM) and the resheating procedure on the target section of the artery (3 passes), simulating the maneuvers for the thrombus capture and retrieval (in absence of clot), and the withdrawal of the device, according to the instructions for use of the devices and the standard procedure provided in the clinical use.

In interventions with ANA combined with the CM, the funnel was expanded proximally, in the proximal segment of the renal artery (large vessel with 3-4.5 mm in diameter), allowing sufficient vessel length to deploy the clot mobilizer distally in the distal branch (small vessel with 2-3 mm in diameter). This allowed the evaluation of the compatibility of the funnel with the CM and the vessel injury caused by both devices. A similar procedure was followed when the tested devices were combined with only a standard guide catheter, but in this case the guide catheter distal end reached the proximal segment of the renal artery, and the CM was deployed distally in the distal branch and retrieved until reaching the proximal section of the renal artery. The general performance of devices including the devices preparative and compatibility, visibility, navigability, flexibility and pushability, withdrawal and devices integrity assessment were semi-qualitatively evaluated.

Additionally, device performance with respect to the assessment of clot retrieval was done in animals C21, C22, C23, C25, C27, C28, C29, C30, C31, C33, C34, C35, C36, C37, C38 and C39. A fragment of an ex vivo radiopaque autologous clot was introduced into the left or right subclavian artery or a branch of the carotid artery (i.e. cervical artery) followed by a test device introduction and thrombectomy maneuver to attempt to retrieve the clot, according to directions for use.

To document the target vessel and the performance of the devices, several angiographies were registered during the intervention. Vascular response, including vasospasm, dissection, perforation or thrombosis, and revascularization was evaluated using angiographies. Angiographic images of the vessel were obtained with contrast media (Lopamidol, 10 ml/kg, 370 mg/ml) to identify the proper location for the deployment site. Additional angiograms may have been recorded at this point, or later on, during the procedure, at the discretion of the interventionalist. Baseline angiogram was performed to document the reference diameter for the device access and deployment. It was verified that the guide catheter was positioned in the target vessel. Baseline angiogram was performed to document the reference diameter for the device access and deployment. Post-procedural angiographies were registered to assess the vessel status and/or revascularization after clot retrieval (mTICI scale, defined in Example 1). Also, the general performance including the compatibility of the ANA with Conda were assessed.

The interventional procedure was carried out as follows:
Vascular Access: After induction of anesthesia, the right femoral artery was accessed through an incision made in the inguinal region. An arterial short 8F sheath was introduced and advanced into the artery, under echographic guidance. A venous blood sample was collected prior to heparin administration and fluid therapy initiation.
Vessel Angiography: Under fluoroscopic guidance, a 6F guide catheter-long sheath (Neuron Max) was inserted through the sheath using a 5F diagnostic catheter Simmons type I and a wire guide, and advanced to the target location: the cranial branch of the left renal artery. Guide catheter was continuously flushed with saline solution. Then, the diagnostic catheter and guide wire was removed.
Clot embolization: A carotid artery branch or subclavian artery (section with a diameter approx. 3-4 mm) was embolized with an ex vivo autologous radiopaque clot of 4-4.5 mm diameter 5-20 mm length (synthetized by personnel in the animal facility VHIR according to internal patented procedure) contained in a 1 ml syringe. A 6F guide catheter was located in the target artery, then the syringe containing the clot was connected and the clot injected distally in the target artery. The guide catheter was removed for 5-10 minutes to reestablish the flow and allow the clot stabilization within the artery lumen. Hereinafter the guide catheter was reintroduced, and an angiography was performed to confirm the vessel occlusion and assess the artery flow (TICI).
Device procedure: The target site of the vessel was reached with the ANA and the CM, the clot was retrieved and all the system was withdrawn outside the body.
Device usability and performance: After the procedure, interventionists provided feedback on the usability and performance of the devices following the endpoints shown in Table 6. Subjective evaluation of devices usability and performance characteristics was based on the judgment and experience of the interventionist according to the rating scale. R&D Anaconda team also provided a qualitative assessment of the device's usability and performance. Recanalization was calculated using mTICI scale before clot removal and after clot removal in subclavian or carotid artery branches.
Radial forces against the wall of the blood vessel: The radial forces were obtained using the equipment RX550 from Machine Solutions Inc. With this equipment the radial force and the diameter while decreasing and/or increasing diameter was measured according to all the stent profile. The specific geometry of the head (12 segments) of the equipment allowed the radial compression of the sample uniformly, with very low friction force. 5 cycles of compression/decompression was done in order to obtain curves defined how the radial forces varies with the diameter.

Additionally, semi-quantitative microscopic artery segments evaluation was performed by applying the following histological endpoints: endothelial denudation, intraluminal and mural thrombus, edema edemas, ruptures, hemorrhage, inflammation or injuries in internal, medial or external layers and stenosis.

Results

The evaluation of the different Conda prototypes in combination with the guide catheter and the ANA showed a successful performance regarding to the preparative, compatibility with ancillary devices, navigability, pushability and control-manipulation of the device to the target vessel, visibility of the funnel of the ANA device and the guide catheter, device withdrawal and hemostasis, similar to the Solitaire. The CM and funnel deployment, accuracy in the target vessel and adaptability to the vessel were correct (distal branch and proximal section of the renal artery, respectively). In general, the combination of the Conda prototypes with the funnel and microcatheter was adequate; Conda prototypes with the support of the microcatheter were easily navigated to reach the target vessel (distal branch of the renal artery).

The visual inspections showed that the integrity of the Conda prototypes, the funnel of the ANA device, the guide catheter and the Solitaire was practically unaltered after the application of 3 passes.

Table 6 summarizes the results of the procedures in renal arteries without clot and the performance and usability of the CMs during the interventions.

TABLE 6
CMs scoring of performance and usability in renal arteries with no clot.

| | Scoring | | | | | |
|---|---|---|---|---|---|---|
| | Conda prototypes | | | | | |
| | ID 19 | ID 20 | ID 25 | ID 34 | ID 45 | Solitaire |
| Performance and usability endpoints | n = 1 | n = 1 | n = 2 | n = 6 | n = 4 | n = 5 |
| Navigability/trackability/flexibility of the | 3 | 3 | 3 | 3 | 3 | 3 |
| clot mobilizer/stent retriever to reach | | | | | | |
| the target vessel | | | | | | |
| [1 poor; 2 moderate-adequate; 3 very | | | | | | |
| good] | | | | | | |
| Pushability of the clot mobilizer/stent | 3 | 3 | 3 | 3 | 3 | 3 |
| retriever to the target vessel. Proximal | | | | | | |
| control of the device | | | | | | |
| [1 poor; 2 moderate-adequate; 3 very | | | | | | |
| good] | | | | | | |
| Clot mobilizer/stent retriever | 3 | 3 | 3 | 3 | 3 | 3 |
| deployment | | | | | | |
| [1 no; 2 partially; 3 yes] | | | | | | |
| Accuracy of clot mobilizer/stent | 2 | 2 | 1 (1) | 2 | 2 | 2 |
| retriever deployment in the target vessel | | | 2 (1) | | | |
| [1 no; 2 yes] | | | | | | |
| Clot mobilizer adaptability to the vessel | 1 | 1 | 1 | 1 | 1 | 1 |
| size and curves. Is the funnel-stent | | | | | | |
| completely deployed [1 yes] or kinked [2 no]? | | | | | | |
| Clot mobilizer/stent retriever integrity | 1 | 3 | 3 | 3 | 3 | 3 |
| after intervention (visual inspection) | | | | | | |
| [1 poor; 2 moderate; 3 good] | | | | | | |

Conda prototypes with the support of the microcatheter were easily navigated to reach the target area; the trackability, flexibility and pushability of the clot mobilizer were assessed correctly and the stent deployment and adaptability to the vessel were correct in all cases, in exception of the Conda ID25 (FIG. 10) which its accuracy was not correct in one intervention. The deployed Conda was pushed back through the vessel until being introduced inside the expanded funnel, once it was inside the aspiration was applied. In exception of Conda ID19 (FIG. 1A), which suffered alterations in their integrity during interventions, all other prototypes presented a good integrity after interventions.

Solitaire performance during the clot retrieval was successful after three passes on pig C23 but not in C25, where problems of interaction with the funnel occurred, all problems were reverted after 2^{nd} or 3^{rd} pass.

Table 7 and Table 8 summarize the results of the procedures in subclavian and carotid artery branches with clot occlusions and the performance and usability of the CMs during the interventions. Conda ID19 (FIG. 1A) and ID20 (FIG. 1B) were not evaluated in subclavian and carotid artery branches.

TABLE 7
Summary of interventions (interv.) of subclavian or carotid artery branches with
clot. Correct = correct performance with no incidences, success to retrieve the clot.

| | | Target | | Nº of | TICI before | TICI after | Performance |
|---|---|---|---|---|---|---|---|
| Interv. | Animal | artery | Device | passes | interv. | interv. | results |
| 1. | C23 | Left | Solitaire + | 1 | 0 | 3 | 1^{st} pass: |
| | | Brachial | ANA | | | | correct |
| 2. | C23 | Left | Solitaire + | 1 | 0 | 3 | 1^{st} pass: |
| | | Brachial | ANA | | | | correct |
| 3. | C29 | Left | ID 25 + | 1 | 0 | 3 | 1^{st} pass: |
| | | Cervical | ANA | | | | correct |
| 4. | C29 | Left | ID 25 + | 1 | 0 | 3 | 1^{st} pass: |
| | | Subclavian | ANA | | | | correct |
| 5. | C29 | Left | ID 25 + | 1 | 0 | 3 | 1^{st} pass: |
| | | Cervica | ANA | | | | correct |
| 6. | C30 | Left | ID 25 + | 3 | 0 | 3 | 3^{rd} pass: |
| | | Subclavian | ANA | | | | correct |
| 7. | C30 | Left | ID 25 + | 1 | 0 | 3 | 1^{st} pass: |
| | | Subclavian | ANA | | | | correct |
| 8. | C33 | Left | ID 34 + | 1 | 0 | 3 | 1^{st} pass: |
| | | Subclavian | ANA | | | | correct |
| 9. | C34 | Left | ID 34 + | 1 | 0 | 3 | 1^{st} pass: |
| | | Subclavian | ANA | | | | correct |
| 10. | C34 | Left | ID 34 + | 1 | 0 | 3 | 1^{st} pass: |
| | | Subclavian | ANA | | | | correct |
| 11. | C35 | Left | ID 34 + | 1 | 0 | 3 | 1^{st} pass: |
| | | Subclavian | ANA | | | | correct |
| 12. | C36 | Left | ID 34 + | 1 | 0 | 3 | 1^{st} pass: a |
| | | Cervical | ANA | | | | little fragment |
| | | | | | | | of the clot |
| | | | | | | | remained in |
| | | | | | | | the artery |
| | | | | | | | after pass, no |
| | | | | | | | attempt was |
| | | | | | | | made to |
| | | | | | | | extract it |
| 13. | C38 | Right | ID 45 + | 1 | 0 | 3 | 1^{st} pass: |
| | | Cervical | ANA | | | | correct |
| 14. | C39 | Left | ID 45 + | 1 | 0 | 3 | 1^{st} pass: |
| | | Cervical | ANA | | | | correct |
| 15. | C39 | Carotid | ID45 + | 1 | 0 | 3 | 1^{st} pass: |
| | | branch | ANA | | | | correct |

ANA funnel was navigated and deployed proximally to the clot. The Conda prototypes with the support of the microcatheter were easily navigated to reach the target area with the clot through the internal lumen of ANA funnel catheter, the stent deployment and adaptability to the vessel was correct. The deployed Conda (with the embedded clot) was pushed back through the vessel until being introduced inside the expanded funnel, once it was inside the aspiration was applied. The clot extraction maneuverability was successful after the first pass in all procedures (TICI 2b-3), with no need of additional passes, except for the Conda ID25 (pig C30) in the left subclavian artery in which three passes were required. The general performance and usability of the Conda prototypes were comparable to procedures in renal arteries.

Solitaire performance during the clot retrieval was successful after 1^{st} pass, even though a distal embolization occurred in the left brachial artery of pig C23.

TABLE 8
CMs scoring of performance and usability in subclavian
and carotid artery branches with clot occlusion.

| | Scores | | | |
|---|---|---|---|---|
| | Conda Prototypes | | | |
| | ID25 | ID34 | ID45 | Solitaire |
| Performance and usability endpoints | n = 5 | n = 5 | n = 3 | n = 2 |
| Navigability/trackability/flexibility of the clot mobilizer/stent | 3 | 3 | 3 | 3 |
| retriever to reach the target vessel | | | | |
| [1 poor; 2 moderate-adequate; 3 very good] | | | | |
| Pushability of the clot mobilizer/stent retriever to the target | 3 | 3 | 3 | 3 |
| vessel. Proximal control of the device | | | | |
| [1 poor; 2 moderate-adequate; 3 very good] | | | | |
| Clot mobilizer/stent retriever deployment | 3 | 3 | 3 | 3 |
| [1 no; 2 partially; 3 yes] | | | | |
| Accuracy of clot mobilizer/stent retriever deployment in the | 2 | 2 | 2 | 2 |
| target vessel [1 no; 2 yes] | | | | |
| Clot mobilizer adaptability to the vessel size and curves. Is | 1 | 1 | 1 | 1 |
| the funnel-stent completely deployed | | | | |
| [1 yes] or kinked [2 no] | | | | |
| Clot mobilizer/stent retriever integrity after intervention | 3 | 3 | 3 | 3 |
| (visual inspection) | | | | |
| [1 poor; 2 moderate; 3 good] | | | | |
| Clot loss during retrieval [1 no; 2 yes] | 1 | 1 | 1 | 1 |
| Recanalization success (thrombus extraction: artery flow | 2 | 2 | 2 | 2 |
| recovery) [1, TICI 0-2a = no/2, TICI 2b-3 = yes] | | | | |
| Clot extraction [1 clot completely out the funnel; 2 clot | 2 | 2 | 2 | 2 |
| completely inside funnel-clot mobilizer/stent retriever; 3 clot | | | | |
| partially inside funnel-clot mobilizer/stent retriever; 4 clot | | | | |
| inside funnel catheter-clot mobilizer/stent retriever] | | | | |

Conda prototypes with the support of the microcatheter were, easily navigated to reach the target area; the trackability, flexibility and pushability of the clot mobilizer were assessed correctly and the stent deployment and adaptability to the vessel were correct in all cases. The deployed Conda was pushed back through the vessel until being introduced inside the expanded funnel, once it was inside the aspiration was applied. No alterations in the integrity of the devices during the interventions were observed.

Solitaire performance during the clot retrieval was successful after three passes on pig C23 but not in C25, where problems of interaction with the funnel occurred, all problems were reverted after the 2^{nd} or 3^{rd} pass.

Conda compatibility with conjunction devices resulted excellent in most of the interventions and the integrity of the clot mobilizer and funnel after intervention resulted good in most of the interventions.

In Table 9, the results of RFs and the vascular damage results on the distal segment of the vessel are presented. The RF were measured at 2 mm because it is the lowest diameter for which the Conda is intended.

TABLE 9
Radial Forces (RF) of the CMs against the wall of the blood vessel.

| Prototype ID | ID19 | ID20 | ID25 | ID34 | ID45 | Solitaire |
|---|---|---|---|---|---|---|
| Average RF at 2 mm [N] | 1.6 | 1.29 | 1.59 | 1.36 | 1.73 | 2.28 |

As is shown in Table 9, the values of RF generated by all Conda prototypes, in relation to the diameter of the vessel, resulted between the maximum limit of 2.0N delimited by the behavior of the Solitaire in the same diameter and the minimum of 1.0N, so the results were inscribed inside the optimal range of vessel diameters (from 1.5 to 3.5 mm).

### CONCLUSIONS

This study showed that Conda in conjunction with ANA obtained better results than Solitaire in conjunction with ANA. On the other hand, Conda showed similar results to Solitaire when they were used in conjunction with BGC. These results imply that the Conda is an effective device for the extraction of clots, and it is the better choice than Solitaire when used together with the ANA device.

Conda also showed very good behavior in reaching the target vessel, being adapted to the vessel wall, and without problems during the retrieval. No damage was found in Conda prototypes due to their use in conjunction with ANA device, confirming that are totally compatible with ANA.

The general performance of the Conda prototypes in combination with ANA was successful and comparable to the Solitaire: device preparative, navigability and pushability. The clot extraction was successful after the first pass with all the Conda prototypes in combination with ANA. The vascular injury in vessels treated with the Conda prototypes in combination with ANA after 3 passes is minimal-mild and comparable to the control device Solitaire combined with ANA.

As final conclusions, ID34 (FIG. 1D) and ID45 (FIG. 1E) obtained the best results and Conda is a clear option to be used in clinical trials to increase the revascularization rate during mechanical thrombectomies.

### Example 3: Radial Force Comparison Between Conda and Solitaire

### Introduction

This test was developed in order to compare the outward radial forces (wall apposition) generated by two stent retrievers (Conda and Solitaire, described in Examples 1 and 2) depending on the lumen diameter of the vessels. After extraction of the data, graphical description (outward radial force vs. vessel diameter) comparing Solitaire and Conda devices, was generated.

Methods

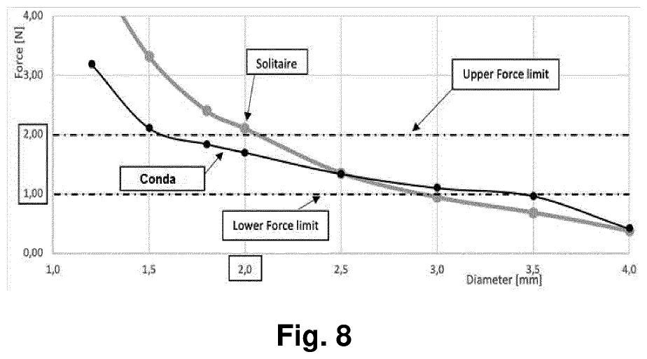

Both devices (samples) were tested for radial force (RF). The RX550 from Machine Solutions Inc. was used to measure both, the RFs and the diameter of the devices. The specific geometry of the head (12 segments) allows compressing the sample uniformly, with very low friction force. RX550 temperature was set to the defined test temperature (37±2° C.). The samples were introduced in the head and profile was started: the RX bench decreases and/or increases the diameter according to specified profile and the RFs and diameter were recorded. Pictures of the sample during the test were taken. At the end of the test, RX550 head was opened and the sample was retrieved from the RX550 then placed within its corresponding container. Lastly, FIG. 8 depicting the individual RF vs. diameter was plotted.

The intended use for the stent retrievers is between 1.5-4.0 mm. RF safety limits were defined starting from the vessel diameter of 2.0 mm which, in Solitaire values, was at 2.0 N. This value assured that there were not endothelial damage and, therefore, it was the maximum value. On the other hand, the minimum value was 1.0 N, which was the minimum value to extract a thrombus in optimal conditions.

Results

As is shown in FIG. 8, the values of RF generated by Conda, in relation to the diameter of the vessel, resulted between the maximum limit of 2.0 N delimited by the behavior of the Solitaire in the same diameter and the minimum of 1.0 N, so the results were inscribed inside the optimal range of vessel diameters (from 1.5 to 3.5 mm), generating a flat curve for a broad range of vessels. The curve provided by the proposed clot mobilizer device 1 resulted much smoother than the curve of Solitaire, which allowed lower RFs in small diameters and larger radial forces in large diameters without, therefore, leaving the limited safety window. Remarkably, due to the fact of having less radial force in small diameters, the risk of damage the vessel is reduced, in comparison with Solitaire. Also, the greatest force in large diameters never exceeded 2.1 N, observed in the case of the Solitaire device for diameters outside the range 2.0-3.0 mm.

### Example 4: Torque Resistance Test of the Attachment (i.e., the Connection) Between the Pusher 200 and the Elongated Device 100 of a Clot Mobilizer Device 1 (in this Particular Case, a Stent Retriever

The purpose of this experimental test was to measure the torque resistance of the attachment between the pusher 200 and the elongated device 100 of a clot mobilizer device 1.

Eight stent retrievers were used in this study: Five stent retrievers manufactured by Anaconda Biomed with different configurations (hereinafter Conda devices, FIGS. 1A-1E) and three manufactured by Medtronic (hereinafter Solitaire devices).

Methods

The tools and equipment used during the procedure are exposed in Table 10.

TABLE 10
Tools and equipment used in Example 4.

| Tools and Equipment | Description |
|---|---|
| Horizontal Tensile Tester | E7451; Used as a surface to correctly place the |
| | fixtures |
| Proximal torque tooling | Fixture used to hold the guide wire torquer and |
| | perform and quantify rotational movement |
| Torque Load Cell | Load cell used to register the torque force |
| Torque Load Cell Holder | Fixture used to place the torque load cell |
| Chuck | Element used to hold the stent compressing |
| | radially |
| Flatbed Chuck Holder | Fixture used to correctly place the chuck |
| Guide wire torquer | Element used to hold the push wire proximally |
| Hypotube with clamps | Element used to maintain the push wire straight |
| Pin Gauge (0.1645″) | Element in which the stent is fitted to avoid its |
| | collapse while is being held in the chuck |

Procedure:

1. The stent retrievers were preconditioned in water at 37° C.±2° C. for at least 2 hours before testing.

2. The 3D printed clamps and proximal torque tooling were set up on the baseboard of the horizontal tensile tester at the following locations and with distal clamp, proximal clamp and proximal torque tooling (placed during setup).

3. The proximal torque tooling and the 3D printed fixture for the proximal torque tooling were placed at the left-hand side of the water bath.

4. The connection portion of the stent retriever was clamped using the guidewire torquer into the jaws of the hand-held torque gauge.

5. The stent retriever was loaded through the hypotube and clamp the distal end of the stent retriever into the Torque Load Cell using a pin gauge.

6. The hand-held torque gauge was set to peak force Nm.

7. The hand-held torque gauge was rotated 360° by allowing the torque gauge holder to click every 90° to aid the operator.

8. The hand-held torque gauge was rotated 360° until a break occurs.

9. When the test was completed, the following details were recorded on a datasheet: Peak Force, Number of Revolutions, Location of Break, Failure Mode.

Results and Conclusion

The following Table 11 shows the results for both, Conda and Solitaire devices:

TABLE 11
Comparison torque results between Conda and Solitaire devices.

| N° of revolutions before break | Conda | Solitaire |
|---|---|---|
| Mean | 31.2 | 22.0 |
| Minimum | 27.0 | 21.0 |
| Maximum | 34.0 | 23.0 |
| Standard deviation | 3.1 | 1.0 |

The results showed a higher performance of the Conda devices during torque test compared to Solitaire devices. These results are directly related with the safety of the clot mobilizer device 1 during the clinical use, therefore, obtaining a higher number of revolutions reduce the possibilities of a premature breakage due to e.g., an incorrect handling of the device. However, the stent retriever devices were not intended for torque during use, so the values obtained for both Conda and Solitaire devices are enough to cover any possible misuse of the clot mobilizer device 1.

### Example 5: Tensile Test of the Attachment (i.e., the Connection) Between the Pusher 200 and the Elongated Device 100 of a Clot Mobilizer Device 1 (in this Particular Case, a Stent Retriever

The purpose of this experimental test was to measure the tensile of the attachment between the pusher 200 and the elongated device 100 of a clot mobilizer device 1.

Thirty-five samples, of them, twenty Conda devices (FIG. 1), and fifteen Solitaire devices, were used as stent retrievers to evaluate the tensile strength in the attachment/connection.

The study was developed by following healthy and safety protocols and when using any equipment described in this procedure to avoid accidental injury around all the procedure, always keeping safe the fingers and limbs during the movement and interaction with tools and equipment.

Methods

The tools and equipment used during the procedure are exposed in Table 12.

TABLE 12
Tools and equipment used in Example 5.

| Tools and | EF-/E- | |
|---|---|---|
| Equipment | Number | Description |
| Zwick Tensile | E11246-01 | Equipment with grips with controlled |
| Tester | or equivalent | displacement in one axis, connected |
| | | to Xpert Zwick software |
| 50N Load Cell | Any Calibrated | Load cell installed in the Tensile |
| | | tester for registering the forces |
| Pneumatic Grips | Record on Use | Grips integrated in the Tensile |
| | | tester with a opening/closing system |
| | | by pressurized air |
| Ruler | Any Calibrated | Used to check the distances and |
| Water Bath | Record on Use | Used to precondition the units to |
| | | 37° C. |
| Thermometer | Any Calibrated | Used to control that the temperature |
| | | in the preconditioning step is |
| | | correctly reached |
| Ceramic grips | N/A | Elements integrated into the |
| | | pneumatic grips that facilitates |
| | | the correct holding of the devices |

Procedure:

1. The samples were preconditioned in water at 37° C.±2° C. for at least 2 hours before testing.

2. The parameters were set up on the connected PC through the Zwick test Xpert program (3104236 Rev 01 Clot Mobiliser System Tensile Test.ZP2), as follows: tensile mode, the speed at 4 m/min and grip to grip separation at 10 mm.

3. The configuration of the equipment was checked, and it was ensured that the 50 N load cell was attached, and both pneumatic grips had the ceramic grips attached.

4. A wire of the pusher was placed in the upper grips until the stent bond was positioned in the center point between both sets of pneumatic clamps and the clamps were closed.

5. The force on the tensile tester was set to zero and then the bottom clamp was closed.

6. At this point, the test was started by clicking the button “start”.

7. When the test was completed, the following details were recorded on a datasheet: peak tensile force to break bond, location of bond break, failure mode.

8. Finally, when the detail recording was finished, the samples were removed, and the clamps opened.

Results and Conclusion

The following Table 13 shows the results for both, Conda and Solitaire devices:

TABLE 13
Comparison tensile results between Conda and Solitaire devices.

| Tensile strength (N) | Conda | Solitaire |
|---|---|---|
| Mean | 13.4 | 11.6 |
| Minimum | 11.2 | 9.8 |
| Maximum | 17.4 | 13.0 |
| Standard deviation | 1.9 | 1.1 |

The results showed a higher performance of the Conda devices in tensile resistance compared to Solitaire devices. These results are related with the safety of the clot mobilizer device 1 during the clinical use, therefore, obtaining higher values of tensile strength reduce the possibilities of a premature breakage of the attachment/connection due to an excessive tension during retrieval.

Unless otherwise indicated, all numbers expressing measurements, conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

Throughout the description and claims the word “comprise” and its variations such as “comprising” are not intended to exclude other technical features, components, or steps. Additional objects, advantages and features of the invention will become apparent to those skilled in the art upon examination of the description or may be learned by practice of the invention. Furthermore, the present invention covers all possible combinations of particular and preferred embodiments described herein.

The present disclosure and/or some other examples have been described in the above. According to descriptions above, various alterations may be achieved. All applications, modifications and alterations required to be protected in the claims may be within the protection scope of the present disclosure.

The scope of the present invention is defined in the following set of claims.

1. A clot mobilizer device for extraction of an occlusion from a blood vessel, comprising:
a working portion (101) comprising a plurality of crowns (110₁ . . . 110ₙ) of cells (111), each cell (111) of the working portion (101) comprising an open area bordered by struts (112), a distal end of each cell (111) in a first crown (110₁) of the plurality of crowns (110₁ . . . 110ₙ) being contiguous with a proximal end of a corresponding cell (111) in a third crown (110₃) of the plurality of crowns (110₁ . . . 110ₙ), a distal end of each cell (111) in a second crown (110₂) of the plurality of crowns (110₁ . . . 110ₙ) being contiguous with a proximal end of a corresponding cell (111) in a fourth crown (110₄) of the plurality of crowns (110₁ . . . 110ₙ), the second crown (110₂) being disposed distal to the first crown (110₁) and proximal to the third crown (110₃), the fourth crown (110₄) being disposed distal to the third crown (110₃), first and second opposite midportions of each cell (111) in each of the first (110₁), second (110₂), third (110₃) and fourth (110₄) crowns each being contiguous with a midportion of an adjacent cell (111) in such crown, wherein the plurality of crowns of cells define a tubular-shaped section forming a cylindrically closed structure; and
a tapered portion (120) extending proximally from a proximal end of the working portion (101), the tapered portion (120) comprising a plurality of tapered portion struts (122), the tapered portion (120) having a smaller diameter at a proximal end than an expanded diameter of the working portion (101), the tapered portion struts (122) having a width greater than a width of the working portion struts (112).

2. The clot mobilizer device of claim 1, wherein the working portion (101) is configured to have a compressed diameter of less than 1.5 mm and to exert an outward radial force between 1.75 N and 3 N when the compressed diameter is around 1.5 mm; and wherein the working portion (101) is configured to have an expanded diameter of at least 3.5 mm and to exert an outward radial force between 0.75 N and 1.5 N when the expanded diameter is around 3.5 mm.

3. The clot mobilizer device of any one of claims 1-2, wherein first and second tapered portion struts (122a, 122b) of the plurality of tapered portion struts (122) converge from a proximal end of the working portion (101) to a distal end (123) of a proximal connection portion (121) to partially define a proximal cell (124), the clot mobilizer further comprising a pusher (200) extending proximally from the proximal connection portion (121).

4. The clot mobilizer device of claim 3, wherein a third tapered portion strut (122c) of the plurality of tapered portion struts (122) extends distally from the first tapered portion strut (122a) from a point distal to the proximal connection portion (121) and a fourth tapered portion strut (122d) of the plurality of tapered portion struts (122) extends distally from the second tapered portion strut (122b) from a point distal to the proximal connection portion (121), the third and fourth tapered struts (122c, 122d) partially defining the proximal cell (124).

5. The clot mobilizer device of claim 4, wherein the third and fourth tapered portion struts (122c, 122d) converge at the proximal end of the working portion (101).

6. The clot mobilizer device of any one of claims 1-5, wherein the cells (111) in the working portion (101) have an almond shape.

7. The clot mobilizer device of any one of claims 1-6, wherein the clot mobilizer device (1) is manufactured by producing specified cuts either on a tube or on a wire.

8. The clot mobilizer device of any one of claims 1-7, wherein the working portion struts (112) have a width between 30 μm and 60 μm and the tapered portion struts (122) have a width between 60 μm and 155 μm.

9. The clot mobilizer device of any of claims 1-8, wherein the working portion (101) in the compressed diameter has a length between 10 mm and 60 mm, the working portion (101) in the expanded diameter has a length between 20 mm and 50 mm, and the tapered portion (120) has a length between 10 mm and 20 mm.

10. The clot mobilizer device of any one of the claims 1-11, wherein the tapered portion struts (122) have a thickness equal to a thickness of the working portion struts (112).

11. The clot mobilizer device of any one of the claims 1-10, further comprising a plurality of radiopaque markers (107) at least at a distal end of the working portion (101), at least one of the plurality of radiopaque markers (107) having a length different from a length of another of the plurality of radiopaque markers (107).

12. The clot mobilizer device of any one of claims 3-11, wherein:
the proximal connection portion (121) having a first attachment surface (102);
the pusher (200) comprises a second connection portion (201) extending distally, the second connection portion (201) having a second attachment surface (202) facing and extending longitudinally aligned to the first attachment surface (102) to define an overlapping portion and to define first and second seams extending longitudinally along first and second lateral extents of the overlapping portion;
the clot mobilizer device (1) further comprising:
a first weld attaching the proximal and second connection portions (121, 201), extending from the first seam toward the second seam; and
a second weld attaching the proximal and second connection portions (121, 201), extending from the second seam toward the first seam.

13. The clot mobilizer device of claim 12, wherein a cross-sectional shape of the proximal connection portion (121) perpendicular to the longitudinal axis is a section of an annulus and a cross-sectional shape of the second connection portion (201) perpendicular to the longitudinal axis is a circle.

14. The clot mobilizer device of any one of claims 12-13, wherein the first weld comprises a plurality of welding points along the first seam and the second weld comprises a plurality of welding points along the second seam.

15. The clot mobilizer device of any one of claims 12-14, further comprising an inner jacket (132) extending around at least part of the overlapping portion, wherein the inner jacket (132) extends proximally from the overlapping portion around at least part of the pusher (200), a radiopaque element (133) disposed at a proximal end of the overlapping portion, wherein the radiopaque element (133) comprises a coil extending around at least part of the pusher (200), and an outer jacket (131) extending around the radiopaque element (133), wherein the outer jacket (131) extends around at least part of the overlapping portion.
